(19) United States
(12) Patent Application Publication
(10) Pub. No.: US 2026/0276648 A1
(43) Pub. Date: Sep. 17, 2026

(54) UPREGULATED METABOLITE FOUND IN BRAIN CANCER

(71) Applicants: UNIVERSITY OF PITTSBURGH - OF THE COMMONWEALTH SYSTEM OF HIGHER EDUCATION, Pittsburgh, PA (US); THE BOARD OF REGENTS OF THE UNIVERSITY OF TEXAS SYSTEM, Austin, TX (US)

(72) Inventors: Kalil G. Abdullah, Pittsburgh, PA (US); Sam McBrayer, Austin, TX (US)

(73) Assignees: UNIVERSITY OF PITTSBURGH - OF THE COMMONWEALTH SYSTEM OF HIGHER EDUCATION, Pittsburgh, PA (US); THE BOARD OF REGENTS OF THE UNIVERSITY OF TEXAS SYSTEM, Austin, TX (US)

(21) Appl. No.: 19/671,561

(22) Filed: May 8, 2026

Related U.S. Application Data
(63) Continuation of application No. PCT/US2024/055161, filed on Nov. 8, 2024.
(60) Provisional application No. 63/597,523, filed on Nov. 9, 2023.

(51) Int. Cl. G01N 33/68; G01N 30/02; G01N 30/72; G01N 30/86; G01N 30/88; G01N 33/70

(52) U.S. Cl. CPC G01N 33/6812; G01N 30/7233; G01N 30/861; G01N 30/88; G01N 33/6848; G01N 33/70; G01N 2030/027; G01N 2030/8818

(57) ABSTRACT

Methods for identifying tumor tissue and determining a margin of a tumor to be resected in a subject are disclosed within. The methods utilize guanidinoacetic acid (GAA) as a tissue biomarker of malignant brain tumors. The presently disclosed subject matter further relates to methods for providing a diagnosis or prognosis about a cancer in a subject.

### CROSS-REFERENCE TO RELATED APPLICATIONS

This application is a continuation of International Patent Application No. PCT/US2024/055161, filed Nov. 8, 2024, which claims priority to U.S. Provisional Application No. 63/597,523, filed Nov. 9, 2023, the contents of both of which are incorporated by reference in their entireties.

### TECHNICAL FIELD

The presently disclosed subject matter relates to the use of guanidinoacetic acid (GAA) as a tissue biomarker of malignant brain tumors. The presently disclosed subject matter further relates to the use of GAA for identifying tumor tissue and determining a margin of a tumor to be resected in a subject in need thereof.

### BACKGROUND

Glioma is the most commonly diagnosed central nervous system malignancy in the United States and its most aggressive forms, high-grade gliomas (HGGs, Grade 4), carry a median overall survival of just 16 months. HGGs encompass two diagnostic categories, which are defined by isocitrate dehydrogenase (IDH) mutational status: 1) Glioblastoma (GBM), IDH-wildtype, Grade 4, and 2) Astrocytoma, IDH-mutant, Grade 4. HGG progression is associated with profound neurologic deficits, including seizures.

Most patients undergo immediate surgical and chemoradiation treatment. Despite intensive treatment regimen, malignant gliomas recur. Treatment for recurrent GBM is predicated on defining true progression, which is difficult to establish through radiographic imaging. Discriminating tumor recurrence from treatment-related pseudoprogression is a key challenge in the management of GBM. Because no reliable noninvasive methods are available to assess tumor progression, invasive surgeries are often required to obtain a tissue diagnosis. Thus, establishing peripheral biomarkers for glioma monitoring is an important unmet clinical need.

Dysregulated metabolism is a hallmark of cancer and a target for anticancer therapy. The discovery of IDH mutations in lower-grade gliomas (LGGs) and the HGGs that arise from this subset of tumors has reshaped understanding of the key role dysregulated metabolism plays in glioma pathogenesis. In IDH-mutant gliomas, point mutations in IDH enzymes enable them to synthesize the oncometabolite (R)-2-hydroxyglutarate [(R)-2HG]. (R)-2HG accumulates to millimolar levels in IDH-mutant tumors, which increases DNA and histone methylation, alters gene expression, and impairs differentiation. Inhibitors of IDH mutant enzymes are being tested for glioma therapy and one such inhibitor, vorasidenib, was recently approved by the FDA for the treatment of Grade 2 IDH-mutant glioma. Additionally, strategies designed to exploit, rather than inhibit, IDH mutant enzyme activity are being developed for glioma therapy. The discovery and therapeutic targeting of (R)-2HG accumulation in IDH-mutant brain tumors provides proof-of-principle that new insights into altered metabolism in glioma can have broad impact. However, the biochemical hallmarks of other glioma subsets, particularly HGGs, are not well defined. There is need to evaluate the biochemical hallmarks of HGGs to identify universal biomarkers of disease and support non-invasive detection of GBM or its recurrence.

For patients who present with a newly suspected HGG, tissue sampling performed by a craniotomy is the standard of care for both biopsy and maximal resection to achieve cytoreduction. Patients then undergo concurrent chemotherapy and radiation. Despite these interventions, the average progression free survival is 9 months after resection and treatment, and recurrence is universal. Diagnosing recurrence is a key challenge in brain tumor management because imaging findings and patient symptoms can be similar between two clinical entities: true progression and pseudoprogression. True progression is defined as tumor recurrence within or around the resection bed and treatment region. Pseudoprogression does not involve tumor recurrence. Instead, it is an effect of radiation and chemotherapy that results in inflamed and friable tissue that has a similar radiographic appearance to tumor growth. The treatment for these two entities differs: surgery and re-irradiation are indicated for true progression whereas anti-angiogenic and corticosteroid agents are indicated to treat pseudoprogression. Currently, differentiating between these clinical states requires an invasive brain surgery to obtain a definitive tissue diagnosis to guide treatment. The ability to distinguish between recurrence and pseudoprogression non-invasively represents a critical unmet need in neuro-oncology.

### SUMMARY OF THE INVENTION

The presently disclosed subject matter provide a method for identifying tumor tissue to be resected in a subject, comprising: collecting one or more samples from a suspected tumor tissue; determining the level of guanidinoacetic acid (GAA) in the one or more samples; comparing the level of GAA in the one or more samples to a reference level; and defining the suspected tumor tissue as tumor or non-tumor based on the level of GAA in the one or more samples. In certain embodiments, the method further comprises resecting the suspected tumor tissue from the subject where the suspected tumor tissue is defined as tumor. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample.

In certain embodiments, the tumor is a glioma. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma. In certain embodiments, the one or more samples comprise tumor cells of the subject. In certain embodiments, the one or more samples comprise non-tumor cells of the subject.

In certain embodiments, the method further comprises determining the level of a second metabolite in the one or more samples selected from the group consisting of creatine, phosphocreatine, and creatinine. In certain embodiments, the method further comprises comparing the level of the second metabolite in the one or more samples to a second reference level. In certain embodiments, the second reference level is the amount of the second metabolite in a non-tumor tissue sample. In certain embodiments, the method further comprises comparing the ratio of GAA to the second metabolite in the one or more samples to a reference ratio. In certain embodiments, the reference ratio is the ratio of GAA to the second metabolite in a non-tumor tissue sample. In certain embodiments, the second metabolite is creatine.

The presently disclosed subject matter further provides a method for determining a margin of a tumor to be resected in a subject, comprising: collecting one or more samples from one or more suspected tumor tissues; determining the level of guanidinoacetate (GAA) in the one or more samples; comparing the level of GAA in the one or more samples to a reference level; and defining the tumor margin by comparing the level of GAA between the one or more suspected tumor tissues. In certain embodiments, the method further comprises resecting the tumor based on the tumor margin. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample.

In certain embodiments, the tumor is a glioma. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma. In certain embodiments, the one or more samples comprise tumor cells of the subject. In certain embodiments, the one or more samples comprise non-tumor cells of the subject.

In certain embodiments, the method further comprises determining the level of a second metabolite in the one or more samples selected from the group consisting of creatine, phosphocreatine, and creatinine. In certain embodiments, the method further comprises comparing the level of the second metabolite in the one or more samples to a second reference level. In certain embodiments, the second reference level is the amount of the second metabolite in a non-tumor tissue sample. In certain embodiments, the method further comprises comparing the ratio of GAA to the second metabolite in the one or more samples to a reference ratio. In certain embodiments, the reference ratio is the ratio of GAA to the second metabolite in a non-tumor tissue sample. In certain embodiments, the second metabolite is creatine.

The presently disclosed subject matter further provides a method for diagnosing a glioma, comprising: determining the level of guanidinoacetic acid (GAA) in one or more samples from a subject; comparing the level of GAA to a reference level; and diagnosing the subject as having a risk of a glioma if the level of the GAA in the one or more samples is higher than the reference level. In certain embodiments, the method further comprises diagnosing the subject as not having a risk of a glioma if the level of the GAA in the one or more samples is lower than the reference level. In certain embodiments, the reference level is the amount of GAA in one or more reference samples collected from a healthy individual. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

The presently disclosed subject matter further provides a method for determining the aggressiveness of a glioma, comprising: determining the level of guanidinoacetic acid (GAA) in one or more samples from a subject; comparing the level of GAA to a reference level; and determining that the subject has a more aggressive glioma if the level of the GAA in the one or more samples is higher than the reference level. In certain embodiments, the method further comprises determining that the subject has a less aggressive glioma if the level of the GAA in the one or more samples is lower than the reference level. In certain embodiments, the reference level is the amount of GAA in one or more reference samples collected from a healthy individual. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

The presently disclosed subject matter further provides a method for determining the grade of a glioma, comprising: determining the level of guanidinoacetic acid (GAA) in one or more samples from a subject; comparing the level of GAA to a reference level; and determining that the subject has a high risk of a higher grade glioma if the level of the GAA in the one or more samples is higher than the reference level. In certain embodiments, the method further comprises determining that the subject has a low risk of a higher grade glioma if the level of the GAA in the one or more samples is lower than the reference level. In certain embodiments, the reference level is the amount of GAA in one or more reference samples collected from a healthy individual. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

The presently disclosed subject matter further provides a method for determining the risk of recurrence in a subject after the subject receives an anti-cancer treatment, comprising: determining the level of guanidinoacetic acid (GAA) in one or more samples from a subject; comparing the level of GAA to a reference level; and determining that the subject has a high risk of recurrence if the level of the GAA in the one or more samples is higher than the reference level. In certain embodiments, the method further comprises determining that the subject has a low risk of recurrence if the level of the GAA in the one or more samples is lower than the reference level. In certain embodiments, the reference level is the amount of GAA in one or more reference samples collected from a healthy individual. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIGS. 1A-1B show shows the metabolic landscape of adult primary and metastatic brain tumors. FIG. 1A shows deep metabolite profiling of 91 brain tissue surgical specimens from adults. Data depict lipid and polar metabolite abundance profiles of HGG, LGG, metastases, and non-malignant brain tissues. Unsupervised hierarchical clustering was used to group samples by biochemical composition. Patient demographic data and molecular features are displayed (top). “Subtype” denotes transcriptionally-defined glioma subclasses. FIG. 1B shows increased GAA abundance is a biochemical hallmark of adult HGG relative to non-malignant brain (“Brain”).

FIGS. 2A-2B show that dysregulated GAA metabolism is a biochemical hallmark of HGG. FIG. 2A shows random forest classification of HGG, LGG, and non-malignant brain (“Brain”) samples. Metabolites are rank-ordered (left) by variable importance scores (mean decrease accuracy). Corresponding levels in each tissue type are shown (right). GPE=glycerophosphoethanolamine. FIG. 2B shows schema of creatine synthesis and consumption pathways. Spont.=spontaneous.

FIGS. 3A-3H show that adult HGGs preferentially accumulate GAA relative to both LGGs and Brain and display a bottleneck in the creatine synthesis pathway at the step of GAA catabolism. FIGS. 3A-3D show levels of GAA (FIG. 3A), creatine (FIG. 3B), phosphocreatine (FIG. 3C), and creatine (FIG. 3D) in HGG, LGG, and non-malignant brain (“Brain”) human tissues. In panel 3A, only index HGG cases are shown. FIG. 3E shows GAA levels in HGG subtypes and non-malignant brain. “Astro, IDH-mut”=Astrocytoma, IDH-mutant, Grade 4. FIG. 3F shows GAA levels in index and recurrent LGGs. FIG. 3G shows that GAA can discriminate between grades of glioma (Grade 2 vs. Grade 4 and Grade 3 vs. Grade 4) by peak intensity. FIG. 3H shows that a tissue sample from a pediatric HGG subtype, diffuse midline glioma, displays elevated GAA relative to Brain. For all panels except FIG. 3F, only index cases are shown. Bars are mean values; * P<0.05, ** P<0.01, *** P<0.0001, n.s.=not significant.

FIGS. 4A-4F show GAA concentrations in brain tissues and indicate that GAA preferentially accumulates in the HGG microenvironment. FIG. 4A shows absolute quantification of GAA in human tissues. “Brain”=brain tissue not invaded by tumor. Brain: n=3. HGG: n=6. Data are mean values +/−range. FIGS. 4B-4E show MRI Brainlab localization of four tissue subregions from the resection of a HGG, IDH-wildtype, grade 4 with corresponding histology. Absolute GAA concentrations are shown for samples of tumor from uninvolved superficial cerebral cortex (FIG. 4B), fluid-linked attenuated inversion recovery (FLAIR) signaling indicative of edema (FIG. 4C), faintly enhancing tumor margin (FIG. 4D), and solid tumor core (FIG. 4E). Scale bars=250 μm. FIG. 4F shows absolute GAA quantification in samples from FIGS. 4B-4E.

FIG. 5A-5C show that GSC lines display a bottleneck in the creatine synthesis pathway and selectively accumulate and secrete GAA. FIG. 5A shows schema of ¹⁵N₄-arginine stable isotope tracing. FIG. 5B shows ¹⁵N₄-arginine labeling of GAA and creatine pools in non-malignant (NHA cells and BJ fibroblasts) and GSC (BT054 and UTSW63) lines at 18 hours. GAA and creatine isotopologues are shown in different shades of grey. As expected, only M+) (unlabeled) and M+2 (two labeled nitrogens) isotopologues were detected. FIG. 5C shows intracellular (left) and secreted (right) GAA levels in NHA and UTSW63 cell cultures 0, 24, or 48 hours after media change. Secreted GAA was measured in conditioned media. Data are +/−SEM; * P<0.05, ** P<0.01.

FIG. 6 shows imbalanced AGAT and GAMT expression in HGG primary samples. Creatine synthesis pathway gene expression is shown in HGG and non-malignant brain specimens from TCGA and GTEx databases, respectively. Specimens are grouped by sample class. “Subtype” denotes transcriptionally-defined glioma subclasses.

FIGS. 7A-7B show that GAA is a metabolic biomarker of Grade 4 glioma. FIGS. 7A-7B show receiver operating characteristic (ROC) analysis evaluating GAA content as a biomarker discriminating Grade 4 glioma and non-malignant brain (brain; FIG. 7A) or Grade 4 glioma and lower-grade glioma (LGG, FIG. 7B) human tissue specimens.

FIG. 8 shows that the GAA-creatine ratio is a metabolic biomarker of Grade 4 glioma. Receiver operating characteristic (ROC) analysis evaluating the GAA:creatine ratio as a biomarker discriminating Grade 4 glioma and non-malignant (brain) human tissue specimens.

### DETAILED DESCRIPTION

The presently disclosed subject matter relates to methods for identifying tumor tissue and determining a margin of a tumor to be resected using guanidinoacetic acid (GAA) as a tissue biomarker. The presently disclosed subject matter further relates to the use of GAA methods for providing a diagnosis or prognosis about a cancer in a subject and for determining the risk of malignant brain tumors or recurrence.

For purposes of clarity of disclosure and not by way of limitation, the detailed description is divided into the following subsections:
1. Definitions;
2. Intraoperative Glioma Biomarkers; and
3. Peripheral Glioma Biomarkers for Non-invasive Detection.

### 1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “cell” refers to any suitable cell for use in the present disclosure, e.g., eukaryotic cells. For example, but not by way of limitation, suitable eukaryotic cells include animal cells, e.g., mammalian cells. In certain embodiments, suitable cells are cultured cells. In certain embodiments, suitable cells are host cells, recombinant cells, and recombinant host cells. In certain embodiments, suitable cells are cell lines obtained or derived from mammalian tissues which are able to grow and survive when placed in media containing appropriate nutrients and/or growth factors.

The terms “expression” or “expresses,” as used herein, refer to transcription and translation occurring within a cell, e.g., mammalian cell. In certain embodiments, the level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

The term “protein” is meant to refer to a sequence of amino acids for which the chain length is sufficient to produce the higher levels of tertiary and/or quaternary structure. This is to distinguish from “peptides” or other small molecular weight polypeptides that do not have such structure. In certain embodiments, the protein herein will have a molecular weight of at least about 15-20 kDa, e.g., about 20 kDa or greater. Examples of proteins encompassed within the definition herein include host cell proteins as well as all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “protein variant” or “polypeptide variant” refers to a protein or polypeptide that comprise modifications and/or truncations compared to a parent or wild type protein or polypeptide. In certain embodiments, a protein variant can differ from the parent protein or wild type protein by at least one amino acid modification, e.g., from about one to about ten amino acid modifications. In certain embodiments, the sequence of a protein variant sequence has at least about 80%, at least about 90%, at least about 95% or at least about at least about 99% identity to a parent or wild type protein sequence. In certain embodiments, a protein variant can differ from another variant of the protein by at least one amino acid modification, e.g., from about one to about ten amino acid modifications. In certain embodiments, the sequence of a protein variant sequence has at least about 80%, at least about 90%, at least about 95% or at least about at least about 99% identity to a different variant of the protein.

As used herein, the term “mutation” refers to a mutation in an amino acid sequence or in a nucleic acid sequence. In certain embodiments, a mutation in an amino acid sequence can be a substitution (replacement), an insertion (addition), or a deletion (truncation) of at least one amino acid in the amino acid sequence. In certain embodiments, a mutation in a nucleic acid sequence can be a substitution (replacement), an insertion (addition), or a deletion (truncation) of at least nucleotide of the nucleic acid sequence.

As used herein, the term “endogenous,” refers to a nucleic acid molecule or polypeptide that is normally expressed in a cell or tissue.

As used herein, the term “exogenous,” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. As used herein, the term “exogenous” nucleic acid refers to a nucleic acid not present in a native wild-type cell; for example, an exogenous nucleic acid can vary from an endogenous counterpart by sequence, by position/location, or both. For clarity, an exogenous nucleic acid can have the same or different sequence relative to its native endogenous counterpart; it can be introduced by genetic engineering into the cell itself or a progenitor thereof, and can optionally be linked to alternative control sequences, such as a non-native promoter or secretory sequence.

As used herein, the term “increase” refers to altering positively by at least about 5%. An alteration can be an increase of about 5%, about 10%, about 25%, about 30%, about 50%, about 75%, about 100% or more.

As used herein, the term “reduce” refers to altering negatively by at least about 5%. An alteration can be a decrease of about 5%, about 10%, about 25%, about 30%, about 50%, about 75% or more, even by about 100%.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

As used herein, the term “inhibitor” refers to a compound or molecule (e.g., small molecule, peptide, peptidomimetic, natural compound, siRNA, anti-sense nucleic acid, aptamer, or antibody) that interferes with (e.g., reduces, prevents, decreases, suppresses, eliminates or blocks) the signaling function of a protein or pathway. An inhibitor can be any compound or molecule that changes any activity of a protein (signaling molecule, any molecule involved with the named signaling molecule or a named associated molecule). Inhibitors also include molecules that indirectly regulate the biological activity of a named protein, by intercepting upstream signaling molecules.

The terms “inhibiting,” “eliminating,” “decreasing,” “reducing” or “preventing,” or any variation of these terms, referred to herein, includes any measurable decrease or complete inhibition to achieve a desired result.

As used herein, the term “disease” refers to any condition or disorder that damages or interferes with the normal function of a cell, tissue, or organ.

A “therapeutically effective amount” of an agent refers to an amount effective, at dosages and for periods of time necessary, to achieve the desired therapeutic or prophylactic result, e.g., treating a glioma in a subject. A therapeutically effective amount can be administered in one or more administrations.

An “individual” or “subject,” as referred to herein, can be a human or non-human subject, such as, but not limited to, a non-human primate, a dog, a cat, a horse, a rodent, a rabbit, etc.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, prolonging survival, preventing recurrence of disease, alleviation of symptoms, diminishment of any direct or indirect pathological consequences of the disease, decreasing the rate of disease progression, amelioration or palliation of the disease state, and remission or improved prognosis.

An “anti-cancer effect” refers to one or more of a reduction in aggregate cancer cell mass, a reduction in cancer cell growth rate, a reduction in cancer progression, a reduction in cancer cell proliferation, a reduction in tumor mass, a reduction in tumor volume, a reduction in tumor cell proliferation, and/or a reduction in tumor growth rate. In certain embodiments, an anti-cancer effect can refer to a complete response, a partial response, a stable disease (without progression or relapse), a response with a later relapse or progression-free survival in a patient diagnosed with cancer.

An “anti-cancer agent,” as used herein, can be any molecule, compound, chemical or composition that has an anti-cancer effect. Anti-cancer agents include, but are not limited to, chemotherapeutic agents, radiotherapeutic agents, cytokines, anti-angiogenic agents, apoptosis-inducing agents, anti-cancer antibodies and/or agents which promote the activity of the immune system.

### 2. Intraoperative Glioma Biomarkers

Gliomas are divided into four grades, depending on the tumor cells' appearance under a microscope and their genetic aberrations; the higher a tumor's grade number, the more severe it is. Grades 1, 2, and 3 are considered lower-grade gliomas (LGGs). Grade 4 gliomas are considered high-grade gliomas (HGGs). HGGs encompass two diagnostic categories, which are defined by isocitrate dehydrogenase (IDH) mutational status: 1) Glioblastoma (GBM), IDH-wildtype, Grade 4, and 2) Astrocytoma, IDH-mutant, Grade 4.

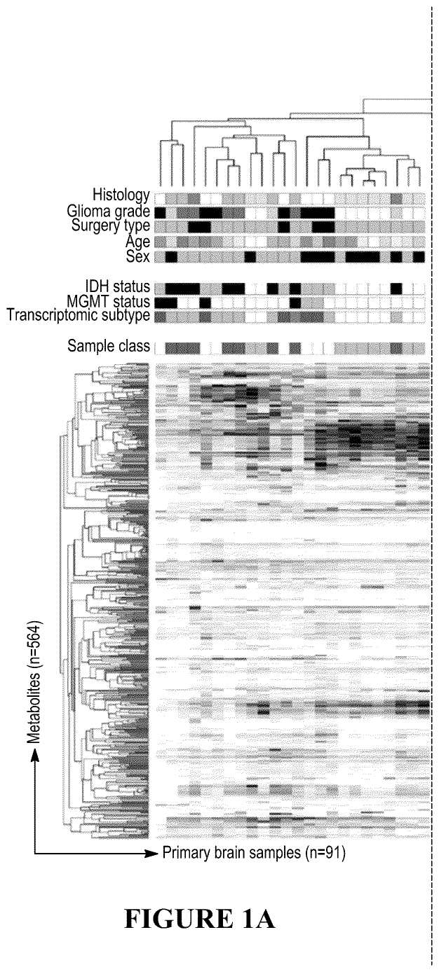

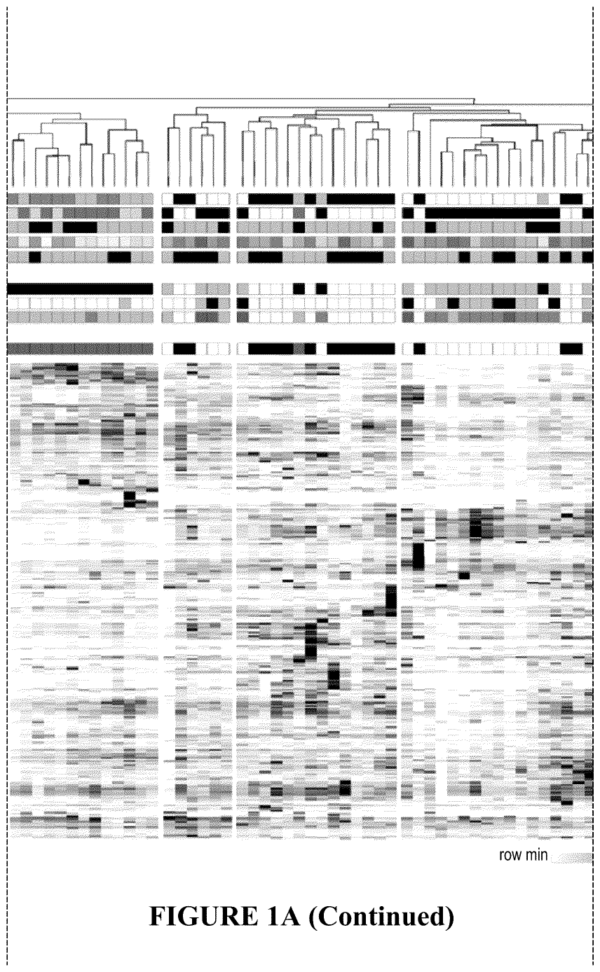

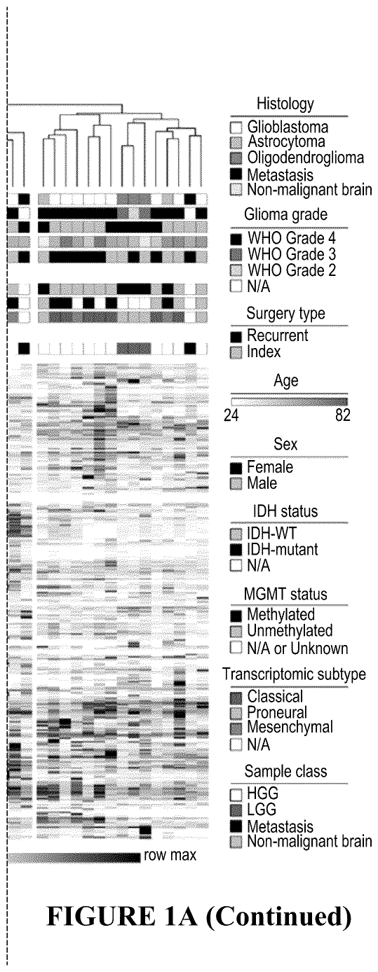

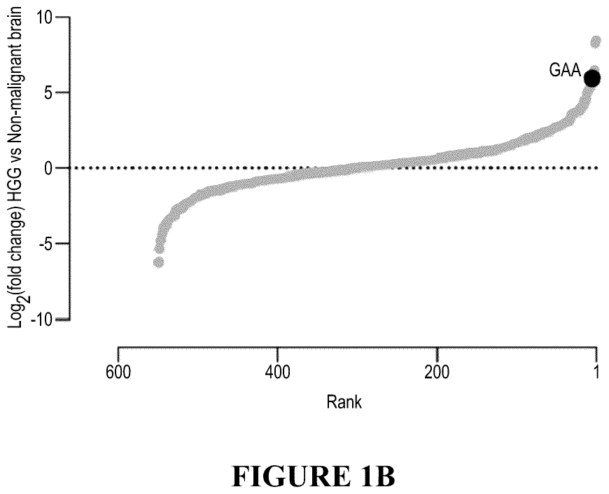

A metabolomics study was performed to identify biomarkers of HGG disease. This study evaluated 564 compounds including guanidinoacetate (GAA) (FIG. 1A and 1B). GAA is an intermediate in the creatine synthesis pathway (FIG. 2B). GAA is produced by the enzyme arginine-glycine amidinotransferase (AGAT) which converts glycine and arginine into GAA and ornithine. GAA is then converted to creatine by the enzyme guanidinoacetate N-methyltransferase (GAMT). The GAMT reaction requires the co-factor S-adenosyl methionine (SAM) which is converted to S-adenosyl-L-homocysteine (SAH). The metabolomics study revealed that guanidinoacetate (GAA) accumulated in HGG tissue, e.g., GBMs, in comparison to non-malignant control tissue (FIG. 2A). This shows that GAA accumulation can be assessed as a biomarker, e.g., for detecting the presence of HGG.

### 2.1 Techniques for Measuring Metabolites During Intracranial Surgery

The presently disclosed subject matter provides methods for determining the level of metabolites, e.g., GAA, in biological samples. The analysis can be used to determine whether the biological sample comprises HGG tissue. The level of metabolites in a sample can be measured using methods known in the art. Metabolites are extracted from samples in a solvent, and metabolite concentrations of extracts are measured. Metabolites can also be measured in tissue aspirates without the need for solvent extraction. Non-limiting methods for determining the level of metabolites, e.g., GAA, in a sample collected from the subject include mass spectrometry. In certain embodiments, the level of GAA in the one or more samples is determined using a mass spectrometry method. In certain embodiments, the mass spectrometry method comprises mass spectrometry (MS), liquid chromatography-mass spectrometry (LC-MS), liquid chromatography with tandem mass spectrometry (LC-MS/MS), or flow injection analysis electrospray ionization tandem mass spectrometry (FIA-ESI-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. GAA precursor and product ions of 118.1 m/z and 76 m/z, respectively, are measured. Isotopically labeled internal standards are included in solvents to enable assessments of extraction efficiency and instrument performance. Absolute GAA quantities are determined by comparing with a range of isotopically labeled GAA standards.

The level of metabolites in a sample can be compared to a reference level. The reference level refers to an amount of metabolite that is relevant for comparison. In certain embodiments, the reference level is a predetermined level of the GAA biomarker. In certain embodiments, the reference level is the level of the GAA biomarker in a healthy individual free of the cancer or a population of healthy individuals free of the cancer. In certain embodiments, the reference is the level of the GAA biomarker in non-tumor cells or non-tumor associated cells.

Biological samples can be collected from a subject during intracranial surgery. The samples can be suspected of comprising HGG tissue or non-malignant tissue. In certain embodiments, the sample include cells of the subject. In certain embodiments, the cells of the subject are tumor cells of the subject. In certain embodiments, the cells of the subject are non-tumor cells of the subject. In certain embodiments, cells to be used with the presently disclosed methods can be obtained by any methods known in the art, including, but not limited to, a surgical resection or a biopsy, for example, needle biopsy, open biopsy, aspirate, or during the resection of a tumor.

In certain embodiments, the methods further comprise determining the level of a second metabolite. As described above, GAA is an intermediate in the creatine synthesis pathway which further includes the conversion of creatine to phosphocreatine and creatinine (FIG. 2B). The level of these or other metabolites in the creatine synthesis pathway can be determined alongside GAA. In certain embodiments, the second metabolite is selected from the group consisting of creatine, phosphocreatine, and creatinine. In certain embodiments, the method comprises comparing the level of the second metabolite in the one or more samples to a second reference level. In certain embodiments, the second reference level is the amount of the second metabolite in a non-tumor tissue sample. In certain embodiments, the method further comprises comparing the ratio of GAA to the second metabolite in the one or more samples to a reference ratio. In certain embodiments, the reference ratio is the ratio of GAA to the second metabolite in a non-tumor tissue sample. In certain embodiments, the second metabolite is creatine.

In certain embodiments, the tumor is a glioma. In certain embodiments, the glioma is a high-grade glioma. In certain embodiments, the glioma is a Grade 4 glioma. In certain embodiments, the glioma is a Grade 3 glioma. In certain embodiments, the glioma is a recurrent glioma. In certain embodiments, the recurrent glioma is a high-grade glioma. In certain embodiments, the recurrent glioma is a Grade 4 glioma. In certain embodiments, the recurrent glioma is a Grade 3 glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

### 2.2 Methods of Tumor Resection Using GAA Biomarker

Tissue samples collected during intracranial surgery can be analyzed according to the methods disclosed herein for the accumulation of metabolites as biomarkers of HGGs. These methods can be used for defining tumor-brain margins during HGG resection surgery by differentiating HGGs and non-malignant tissues based on the accumulation of metabolites, e.g., GAA. Tissues exhibiting GAA accumulation, e.g., HGGs, can be resected while tissues not exhibiting GAA accumulation, e.g., non-malignant tissue, can be left intact.

The presently disclosed subject matter provides methods for identifying tumor tissue to be resected in a subject, comprising collecting one or more samples from a suspected tumor tissue; determining the level of GAA in the one or more samples; comparing the level of GAA in the one or more samples to a reference level; and defining the suspected tumor tissue as tumor or non-tumor based on the level of GAA in the one or more samples. In certain embodiments, the method further comprises resecting the suspected tumor tissue from the subject where the suspected tumor tissue is defined as tumor.

The presently disclosed subject matter further provides methods for determining a margin of a tumor to be resected in a subject, comprising collecting one or more samples from one or more suspected tumor tissues; determining the level of GAA in the one or more samples; comparing the level of GAA in the one or more samples to a reference level; and defining the tumor margin by comparing the level of GAA between the one or more suspected tumor tissues. Additionally or alternatively, the methods disclosed herein comprise identifying the one or more samples of the one or more suspected tumor tissues as tumor, identifying the one or more samples of the one or more suspected tumor tissues as non-tumor, and defining the tumor margin based on the one or more suspected tumor tissues identified as tumor and non-tumor. In certain embodiments, the methods disclosed herein comprise identifying the one or more samples of the one or more suspected tumor tissues as tumor, and defining the tumor margin based on the one or more suspected tumor tissues identified as tumor. In certain embodiments, the methods disclosed herein comprise identifying the one or more samples of the one or more suspected tumor tissues as non-tumor, and defining the tumor margin based on the one or more suspected tumor tissues identified as non-tumor. In certain embodiments, the method further comprises resecting the tumor based on the tumor margin. The presently disclosed subject matter further provides methods for determining a margin of an HGG to be resected in a subject, comprising collecting one or more samples from one or more suspected tumor tissues; determining the level of GAA in the one or more samples; comparing the level of GAA in the one or more samples to a reference level; and defining the tumor margin by comparing the level of GAA between the one or more suspected tumor tissues. Additionally or alternatively, the methods disclosed herein comprise identifying the one or more samples of the one or more suspected tumor tissues as HGG, identifying the one or more samples of the one or more suspected tumor tissues as non-HGG, and defining the tumor margin based on the one or more suspected tumor tissues identified as HGG and non-HGG. In certain embodiments, the methods disclosed herein comprise identifying the one or more samples of the one or more suspected tumor tissues as HGG, and defining the tumor margin based on the one or more suspected tumor tissues identified as HGG. In certain embodiments, the methods disclosed herein comprise identifying the one or more samples of the one or more suspected tumor tissues as non-HGG, and defining the tumor margin based on the one or more suspected tumor tissues identified as non-HGG. In certain embodiments, the method further comprises resecting the tumor based on the tumor margin.

The presently disclosed subject matter further provides methods for resecting tumor tissue in a subject or identifying tumor tissue to be resected in a subject, comprising collecting one or more samples from one or more suspected tumor tissues; determining the level of GAA in the one or more samples; comparing the level of GAA in the one or more samples to a reference level; identifying the one or more suspected tumor tissues as tumor based on the level of GAA. In certain embodiments, the method further comprises resecting the one or more suspected tumor tissues identified as tumor. In certain embodiments, the tumor tissue is an HGG.

The presently disclosed subject matter further provides methods for resecting HGG tissue in a subject or identifying HGG tissue to be resected in a subject, comprising collecting one or more samples from one or more suspected tumor tissues; determining the level of GAA in the one or more samples; comparing the level of GAA in the one or more samples to a reference level; identifying the one or more suspected tumor tissues as HGG based on the level of GAA. In certain embodiments, the method further comprises resecting the one or more suspected tumor tissues identified as HGG.

The level of metabolites in a sample can be measured using methods known in the art. Metabolites can be measured in extracts prepared from samples or in tissue aspirates. Non-limiting methods for determining the level of metabolites, e.g., GAA, in a sample collected from the subject include mass spectrometry. In certain embodiments, the level of GAA in the one or more samples is determined using a mass spectrometry method. In certain embodiments, the mass spectrometry method comprises mass spectrometry (MS), liquid chromatography-mass spectrometry (LC-MS), liquid chromatography with tandem mass spectrometry (LC-MS/MS), or flow injection analysis electrospray ionization tandem mass spectrometry (FIA-ESI-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm.

The level of metabolites in a sample can be compared to a reference level. The reference level refers to an amount of metabolite that is relevant for comparison. In certain embodiments, the reference level is a predetermined level of the GAA biomarker. In certain embodiments, the reference level is the level of the GAA biomarker in a healthy individual free of the cancer or a population of healthy individuals free of the cancer. In certain embodiments, the reference is the level of the GAA biomarker in non-tumor cells or non-tumor associated cells.

Biological samples can be collected from a subject during intracranial surgery. The samples can be suspected of comprising HGG tissue or non-malignant tissue. In certain embodiments, the sample include cells of the subject. In certain embodiments, the cells of the subject are tumor cells of the subject. In certain embodiments, the cells of the subject are non-tumor cells of the subject. In certain embodiments, cells to be used with the presently disclosed methods can be obtained by any methods known in the art, including, but not limited to, a surgical resection or a biopsy, for example, a needle biopsy, core biopsy, or aspirate.

In certain embodiments, the methods further comprise determining the level of a second metabolite. As described above, GAA is an intermediate in the creatine synthesis pathway which further includes the conversion of creatine to phosphocreatine and creatinine (FIG. 2B). The level of these or other metabolites in the creatine synthesis pathway can be determined alongside GAA. In certain embodiments, the second metabolite is selected from the group consisting of creatine, phosphocreatine, and creatinine. In certain embodiments, the method comprises comparing the level of the second metabolite in the one or more samples to a second reference level. In certain embodiments, the second reference level is the amount of the second metabolite in a non-tumor tissue sample. In certain embodiments, the method further comprises comparing the ratio of GAA to the second metabolite in the one or more samples to a reference ratio. In certain embodiments, the reference ratio is the ratio of GAA to the second metabolite in a non-tumor tissue sample. In certain embodiments, the second metabolite is creatine.

In certain embodiments, the tumor is a glioma. In certain embodiments, the glioma is a high-grade glioma. In certain embodiments, the glioma is a Grade 4 glioma. In certain embodiments, the glioma is a Grade 3 glioma. In certain embodiments, the glioma is a recurrent glioma. In certain embodiments, the recurrent glioma is a high-grade glioma. In certain embodiments, the recurrent glioma is a Grade 4 glioma. In certain embodiments, the recurrent glioma is a Grade 3 glioma. In certain embodiments, the glioma is an adult glioma.

In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

### 3. Peripheral Glioma Biomarkers for Non-Invasive Detection

Non-invasive methods for assessing HGG progression are not available. Thus, invasive surgeries are often required to obtain a tissue diagnosis. Establishing peripheral biomarkers for detecting HGG through non-invasive methods is an unmet clinical need. GAA accumulates in blood plasma, cerebrospinal fluid, and urine; thus, GAA accumulation can be assessed as a peripheral biomarker, e.g., for detecting the presence of HGG.

### 3.1 Methods of Diagnosing- and Determining Prognosis of Cancer

GAA accumulation can be evaluated for a subject suspected of having a glioma, e.g., a HGG, using the non-invasive methods disclosed herein. The results can be used to determine the diagnosis, prognosis, and/or aggressiveness of a suspected glioma in a subject without undergoing surgery.

The present disclosure provides methods for diagnosing a glioma, comprising determining the level of GAA in one or more samples from a subject; comparing the level of GAA to a reference level; and diagnosing the subject as having a risk of a glioma if the level of the GAA in the one or more samples is higher than the reference level. In certain embodiments, the method further comprises diagnosing the subject as not having a risk of a glioma if the level of the GAA in the one or more samples is lower than the reference level.

The present disclosure further provides methods for determining the aggressiveness of a glioma, comprising determining the level of GAA in one or more samples from a subject; comparing the level of GAA to a reference level; and determining that the subject has a more aggressive glioma if the level of the GAA in the one or more samples is higher than the reference level. In certain embodiments, the method further comprises determining that the subject has a less aggressive glioma if the level of the GAA in the one or more samples is lower than the reference level.

The present disclosure further provides methods for determining the grade of a glioma, comprising determining the level of GAA in one or more samples from a subject; comparing the level of GAA to a reference level; and determining that the subject has a high risk of a higher grade glioma if the level of the GAA in the one or more samples is higher than the reference level. In certain embodiments, the method further comprises determining that the subject has a low risk of a higher grade glioma if the level of the GAA in the one or more samples is lower than the reference level.

GAA accumulation can be assessed from one or more samples obtained from a subject through non-invasive methods. In certain embodiments, the one or more samples are collected from at least one selected from the group consisting of tumor tissue, cerebrospinal fluid, blood plasma, urine, and combinations thereof.

Low risk of an aggressive tumor is correlated to the survival of the subject. In certain embodiments, the low risk of an aggressive tumor is correlated to the survival of the subject within 1, 2, 3, 4, or 5 years. High risk of an aggressive tumor is correlated to the non-survival of the subject. In certain embodiments, the high risk of an aggressive tumor is correlated to the non-survival of the subject within 1, 2, 3, 4, or 5 years.

According to the methods disclosed herein, the level of GAA in the one or more samples is compared to a reference level. The reference level refers to an amount of GAA that is relevant for comparison. In certain embodiments, the reference level is a predetermined level of the GAA biomarker. In certain embodiments, the reference level is the level of the GAA biomarker in a healthy individual free of the cancer or a population of healthy individuals free of the cancer. In certain embodiments, the reference level is the level of the GAA biomarker in the same subject at an earlier timepoint. In certain embodiments, the reference is the level of the GAA biomarker in non-tumor cells or non-tumor associated cells.

In certain embodiments, the tumor is a glioma. In certain embodiments, the glioma is a high-grade glioma. In certain embodiments, the glioma is a Grade 4 glioma. In certain embodiments, the glioma is a Grade 3 glioma. In certain embodiments, the glioma is a recurrent glioma. In certain embodiments, the recurrent glioma is a high-grade glioma. In certain embodiments, the recurrent glioma is a Grade 4 glioma. In certain embodiments, the recurrent glioma is a Grade 3 glioma. In certain embodiments, the glioma is an adult glioma.

In certain embodiments, the glioma is a pediatric glioma. In certain embodiments, the pediatric glioma is a pediatric astrocytoma, a pediatric oligodendroglioma, or a pediatric diffuse midline glioma.

### 3.2 Methods of Monitoring a Subject's Responsiveness to an Anti-Cancer Treatment

GAA accumulation can be evaluated for a subject suspected of having a recurrent glioma using the non-invasive methods disclosed herein. Recurrent glioma can be suspected in subjects who previously were diagnosed with glioma, e.g., HGG or LGG, and/or received anti-cancer treatments.

The present disclosure further provides methods for determining the risk of recurrence in a subject after the subject receives an anti-cancer treatment, comprising determining the level of guanidinoacetic acid (GAA) in one or more samples from a subject; comparing the level of GAA to a reference level; and determining that the subject has a high risk of recurrence if the level of the GAA in the one or more samples is higher than the reference level. In certain embodiments, the method further comprises determining that the subject has a low risk of recurrence if the level of the GAA in the one or more samples is lower than the reference level.

Non-limiting exemplary anti-cancer treatments that can be used with the presently disclosed methods include, surgical resection, chemotherapy, radiation therapy, targeted drug therapy, immunotherapy, immunomodulatory agents, hematopoietic growth factors, cytokines, monoclonal and polyclonal antibodies, and any combinations thereof. Any suitable chemotherapy or radiotherapy known in the art can be used with the presently disclosed methods. In certain embodiments, the chemotherapy includes administering to the subject a chemotherapeutic agent selected from cisplatin, carboplatin, docetaxel, gemcitabine, paclitaxel, paclitaxel, vinorelbine, pemetrexed, analogues and derivative thereof, and combinations thereof. In certain embodiments, the radiotherapy includes administering to the subject a radiation therapy selected from external beam radiation therapy, stereotactic body radiation therapy, intensity modulated radiation therapy, stereotactic radiosurgery, proton beam therapy, whole brain radiation, or combinations thereof.

Non-limiting methods for determining the level of GAA in a sample collected from the subject include mass spectrometry. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the one or more samples are collected from at least one biological sample selected from the group consisting of tumor tissue, cerebrospinal fluid, blood plasma, urine, and combinations thereof.

According to the methods disclosed herein, the level of GAA in the one or more samples is compared to a reference level. The reference level refers to an amount of GAA that is relevant for comparison. In certain embodiments, the reference level is a predetermined level of the GAA biomarker. In certain embodiments, the reference level is the level of the GAA biomarker in a healthy individual free of the cancer or a population of healthy individuals free of the cancer. In certain embodiments, the reference level is the level of the GAA biomarker in the same subject at an earlier timepoint. In certain embodiments, the reference is the level of the GAA biomarker in non-tumor cells or non-tumor associated cells.

In certain embodiments, the tumor is a glioma. In certain embodiments, the glioma is a high-grade glioma. In certain embodiments, the glioma is a Grade 4 glioma. In certain embodiments, the glioma is a Grade 3 glioma. In certain embodiments, the glioma is a recurrent glioma. In certain embodiments, the recurrent glioma is a high-grade glioma. In certain embodiments, the recurrent glioma is a Grade 4 glioma. In certain embodiments, the recurrent glioma is a Grade 3 glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

### Exemplary Embodiments

The presently disclosed subject matter provide a method for identifying tumor tissue to be resected in a subject. In certain embodiments, the method comprises collecting one or more samples from a suspected tumor tissue. In certain embodiments, the method further comprises determining the level of guanidinoacetic acid (GAA) in the one or more samples. In certain embodiments, the method further comprises comparing the level of GAA in the one or more samples to a reference level. In certain embodiments, the method further comprises defining the suspected tumor tissue as tumor or non-tumor based on the level of GAA in the one or more samples. In certain embodiments, the method comprises: collecting one or more samples from a suspected tumor tissue; determining the level of guanidinoacetic acid (GAA) in the one or more samples; comparing the level of GAA in the one or more samples to a reference level; and defining the suspected tumor tissue as tumor or non-tumor based on the level of GAA in the one or more samples. In certain embodiments, the method further comprises resecting the suspected tumor tissue from the subject where the suspected tumor tissue is defined as tumor. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the tumor is a glioma. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma. In certain embodiments, the one or more samples comprise tumor cells of the subject. In certain embodiments, the one or more samples comprise non-tumor cells of the subject. In certain embodiments, the method further comprises determining the level of a second metabolite in the one or more samples selected from the group consisting of creatine, phosphocreatine, and creatinine. In certain embodiments, the method further comprises comparing the level of the second metabolite in the one or more samples to a second reference level. In certain embodiments, the second reference level is the amount of the second metabolite in a non-tumor tissue sample. In certain embodiments, the method further comprises comparing the ratio of GAA to the second metabolite in the one or more samples to a reference ratio. In certain embodiments, the reference ratio is the ratio of GAA to the second metabolite in a non-tumor tissue sample. In certain embodiments, the second metabolite is creatine.

The presently disclosed subject matter further provides a method for determining a margin of a tumor to be resected in a subject. In certain embodiments, the method comprises collecting one or more samples from one or more suspected tumor tissues. In certain embodiments, the method further comprises determining the level of guanidinoacetate (GAA) in the one or more samples. In certain embodiments, the method further comprises comparing the level of GAA in the one or more samples to a reference level. In certain embodiments, the method further comprises and defining the tumor margin by comparing the level of GAA between the one or more suspected tumor tissues. In certain embodiments, the method comprises: collecting one or more samples from one or more suspected tumor tissues; determining the level of guanidinoacetate (GAA) in the one or more samples; comparing the level of GAA in the one or more samples to a reference level; and defining the tumor margin by comparing the level of GAA between the one or more suspected tumor tissues. In certain embodiments, the method further comprises resecting the tumor based on the tumor margin. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the tumor is a glioma. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma. In certain embodiments, the one or more samples comprise tumor cells of the subject. In certain embodiments, the one or more samples comprise non-tumor cells of the subject. In certain embodiments, the method further comprises determining the level of a second metabolite in the one or more samples selected from the group consisting of creatine, phosphocreatine, and creatinine. In certain embodiments, the method further comprises comparing the level of the second metabolite in the one or more samples to a second reference level. In certain embodiments, the second reference level is the amount of the second metabolite in a non-tumor tissue sample. In certain embodiments, the method further comprises comparing the ratio of GAA to the second metabolite in the one or more samples to a reference ratio. In certain embodiments, the reference ratio is the ratio of GAA to the second metabolite in a non-tumor tissue sample. In certain embodiments, the second metabolite is creatine.

The presently disclosed subject matter further provides a method for diagnosing a glioma. In certain embodiments, the method comprises determining the level of guanidinoacetic acid (GAA) in one or more samples from a subject. In certain embodiments, the method further comprises comparing the level of GAA to a reference level. In certain embodiments, the method further comprises diagnosing the subject as having a risk of a glioma if the level of the GAA in the one or more samples is higher than the reference level. In certain embodiments, the method comprises: determining the level of guanidinoacetic acid (GAA) in one or more samples from a subject; comparing the level of GAA to a reference level; and diagnosing the subject as having a risk of a glioma if the level of the GAA in the one or more samples is higher than the reference level. In certain embodiments, the method further comprises diagnosing the subject as not having a risk of a glioma if the level of the GAA in the one or more samples is lower than the reference level. In certain embodiments, the reference level is the amount of GAA in one or more reference samples collected from a healthy individual. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

The presently disclosed subject matter further provides a method for determining the aggressiveness of a glioma. In certain embodiments, the method comprises determining the level of guanidinoacetic acid (GAA) in one or more samples from a subject. In certain embodiments, the method further comprises comparing the level of GAA to a reference level. In certain embodiments, the method further comprises determining that the subject has a more aggressive glioma if the level of the GAA in the one or more samples is higher than the reference level. In certain embodiments, the method comprises: determining the level of guanidinoacetic acid (GAA) in one or more samples from a subject; comparing the level of GAA to a reference level; and determining that the subject has a more aggressive glioma if the level of the GAA in the one or more samples is higher than the reference level. In certain embodiments, the method further comprises determining that the subject has a less aggressive glioma if the level of the GAA in the one or more samples is lower than the reference level. In certain embodiments, the reference level is the amount of GAA in one or more reference samples collected from a healthy individual. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

The presently disclosed subject matter further provides a method for determining the grade of a glioma. In certain embodiments, the method comprises comprising determining the level of guanidinoacetic acid (GAA) in one or more samples from a subject. In certain embodiments, the method further comprises comparing the level of GAA to a reference level. In certain embodiments, the method further comprises determining that the subject has a high risk of a higher grade glioma if the level of the GAA in the one or more samples is higher than the reference level. In certain embodiments, the method comprises: determining the level of guanidinoacetic acid (GAA) in one or more samples from a subject; comparing the level of GAA to a reference level; and determining that the subject has a high risk of a higher grade glioma if the level of the GAA in the one or more samples is higher than the reference level. In certain embodiments, the method further comprises determining that the subject has a low risk of a higher grade glioma if the level of the GAA in the one or more samples is lower than the reference level. In certain embodiments, the reference level is the amount of GAA in one or more reference samples collected from a healthy individual. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

The presently disclosed subject matter further provides a method for determining the risk of recurrence in a subject after the subject receives an anti-cancer treatment. In certain embodiments, the method comprises determining the level of guanidinoacetic acid (GAA) in one or more samples from a subject. In certain embodiments, the method further comprises comparing the level of GAA to a reference level. In certain embodiments, the method further comprises determining that the subject has a high risk of recurrence if the level of the GAA in the one or more samples is higher than the reference level. In certain embodiments, the method comprises determining the level of guanidinoacetic acid (GAA) in one or more samples from a subject; comparing the level of GAA to a reference level; and determining that the subject has a high risk of recurrence if the level of the GAA in the one or more samples is higher than the reference level. In certain embodiments, the method further comprises determining that the subject has a low risk of recurrence if the level of the GAA in the one or more samples is lower than the reference level. In certain embodiments, the reference level is the amount of GAA in one or more reference samples collected from a healthy individual. In certain embodiments, the level of GAA in the one or more samples is determined using a mass spectrometry method. In certain embodiments, the mass spectrometry method comprises mass spectrometry (MS), liquid chromatography-mass spectrometry (LC-MS) or liquid chromatography with tandem mass spectrometry (LC-MS/MS). In certain embodiments, the level of GAA in the one or more samples is determined using a non-invasive method. In certain embodiments, the non-invasive method comprises magnetic resonance spectroscopy. In certain embodiments, the non-invasive method comprises magnetic resonance spectroscopy, with a singlet peak in the region of 3.78 ppm, or as a ratio between the GAA peak and the nearby Cr peak at 3.19 ppm. In certain embodiments, the reference level is the amount of GAA in a non-tumor tissue sample. In certain embodiments, the glioma is a high-grade glioma (HGG). In certain embodiments, the HGG is a glioblastoma multiforme or astrocytoma. In certain embodiments, the glioma is a Grade 3 glioma. In certain embodiments, the glioma is a Grade 4 glioma. In certain embodiments, the glioma is a recurrent glioma. In certain embodiments, the glioma is an adult glioma. In certain embodiments, the glioma is a pediatric glioma. In certain embodiments, the pediatric glioma is a pediatric astrocytoma, a pediatric oligodendroglioma, a pediatric diffuse midline glioma, or a pediatric diffuse hemispheric glioma.

### EXAMPLES

The present disclosure will be better understood by reference to the following Examples, which are provided as exemplary of the presently disclosed subject matter, and not by way of limitation.

### Example 1: Use of Guanidinoacetate (GAA) as a Biomarker for Malignant Brain Tumors

The biochemical hallmarks of HGG are not well defined. Deeper understanding of HGG metabolism inform new approaches for detecting, monitoring, or treating these tumors. Furthermore, developing biomarkers would greatly enhance clinical monitoring of disease progression and limit surgical interventions in patients with pseudoprogression.

### Methods

Tissue Specimens. Metabolite profiles were collected, annotated, and analyzed from 91 adult surgical brain tissue specimens. These included 39 high grade gliomas (HGGs), 25 lower grade gliomas (LGGs), 18 metastases, and 9 non-malignant brain tissue samples. Gliomas are stratified by both their isocitrate dehydrogenase (IDH) status and their WHO Grade, which range from Grade 1-4. LGGs consist of grades 1-3 gliomas, most of which carry the IDH mutation. HGGs consists of grade 4 tumors which are either mutant or wild type for the IDH mutation. 35 of 39 HGGs were GBMs which are Grade 4 IDH-wildtype tumors and also the most prevalent form of glioma in adult patients worldwide. Importantly, this collection included two control groups: metastases and non-malignant brain tissues whose inclusion enabled identification of biochemical patterns associated with malignancy (by comparing gliomas and non-malignant brain tissues) and cell-of-origin (by comparing gliomas and metastases). Whole exome and RNA sequencing were performed on all specimens to enable integrative multi-omics analyses.

Surgical Technique. Patients electing to undergo surgical resection of brain tumors via craniotomy underwent standard of care preoperative imaging for stereotactic navigation. Intraoperative registration of surface landmarks for frameless stereotaxy was completed, and surgical exposure for a standard of care craniotomy was performed. During the intracranial approach to resection of tumors, samples were isolated from areas that otherwise would be resected, in a manner consistent with an IRB approved study, and these samples were further processed in a sterile field. Each sample location was annotated using intraoperative stereotaxy and a screenshot was taken on a secure server, then processed in a de-identified, HIPAA compliant manner. The individual samples were obtained with a cupped forceps, and then gently placed on a sterile gauze. Samples were sectioned in two equal pieces using a #11 blade scalpel. One section was immediately snap frozen in liquid nitrogen for LC-MS analysis, and the other immediately fixed in formalin for histopathological correlation.

Liquid chromatography-mass spectrometry (LC-MS). Metabolites were quantified from human brain tissues and HGGs using LC-MS. Isotopically labeled internal standards included in solvents were used to enable assessments of extraction efficiency and instrument performance. Absolute quantities were determined by comparing with a range of isotopically labeled standards.

### Results

GAA accumulation is a metabolic hallmark of HGG. The metabolomics study comprised 564 compounds (both polar metabolites and lipids). Unsupervised hierarchical clustering of the metabolomics dataset revealed two dominant clusters stratified largely by malignant status: one cluster was enriched in non-malignant brain and LGG tissues, whereas the other was enriched in HGG samples and metastases (FIGS. 1A and 1B). Subclusters within these groups were strongly associated with cell of origin differences and established glioma-specific gene expression modules.

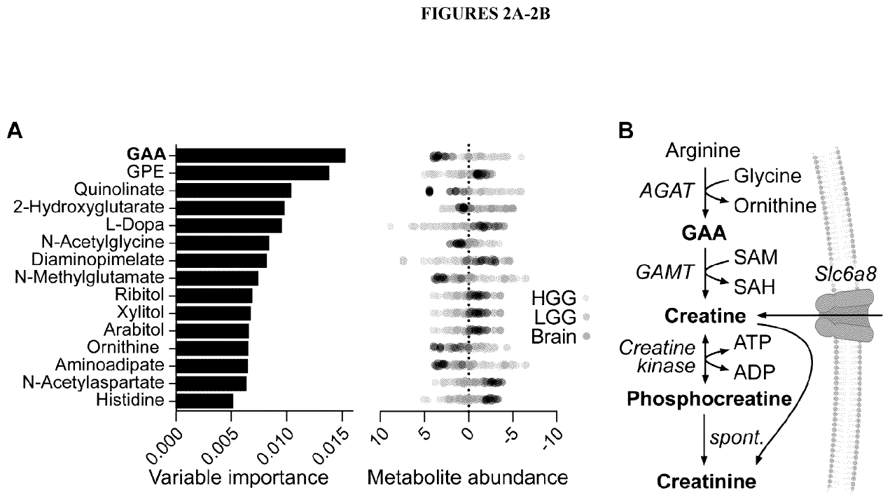

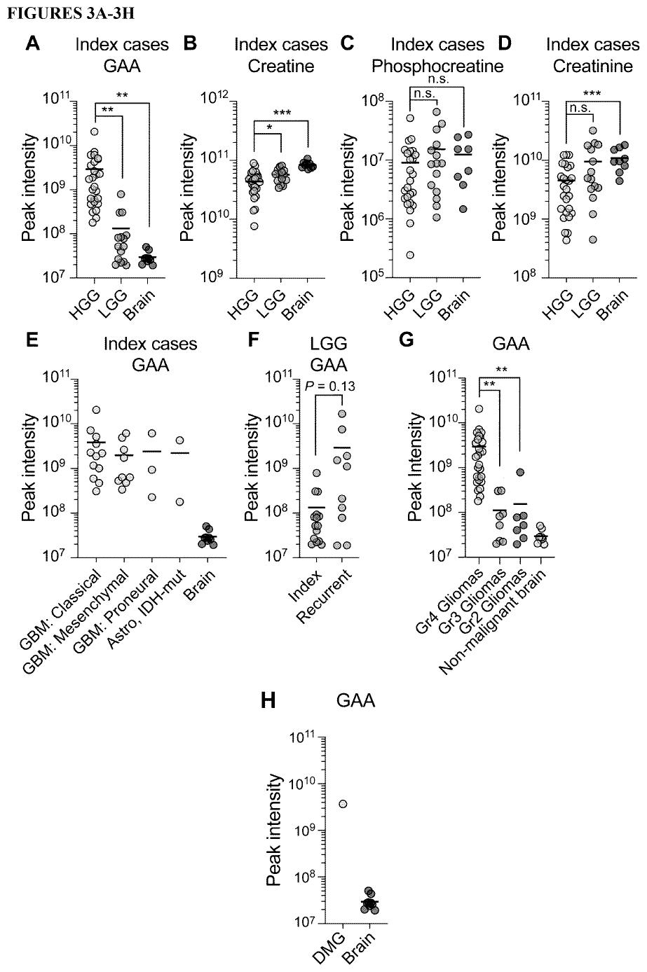

Metabolic hallmarks of adult primary brain tissue subtypes were evaluated using random forest classification of gliomas and non-malignant brain specimens. The compound 2-hydroxyglutarate (2-HG), the product of isocitrate dehydrogenase (IDH) mutant enzymes, was the fourth most important variable for tissue classification and was enriched in IDH-mutant LGGs, thus supporting the validity of the dataset (FIG. 2A). Unexpectedly, one of the most upregulated metabolites in adult HGG relative to non-malignant brain tissue was GAA, an intermediate in the creatine synthesis pathway (FIG. 2B) produced by the enzyme arginine-glycine amidinotransferase (AGAT). GAA was elevated 98-fold in HGGs versus non-malignant brain specimens (FIG. 3A). Interestingly, distal metabolites in this pathway showed an opposite pattern. Creatine and creatinine were downregulated in HGGs relative to non-malignant brain tissue and phosphocreatine showed a similar, albeit statistically insignificant, trend (FIGS. 3B-3D). GAA was upregulated across all HGG subtypes, including all transcriptional subtypes of GBM which comprised most HGG samples (FIG. 3E), and trended higher in recurrent versus index LGG samples (FIG. 3F). GAA was significantly elevated in Grade 4 glioma versus Grade 2 or Grade 3 gliomas (FIG. 3G). GAA was also significantly elevated in a specimen from a pediatric HGG subtype, diffuse midline glioma (DMG, FIG. 3H). These findings link GAA accumulation with aggressive disease in glioma.

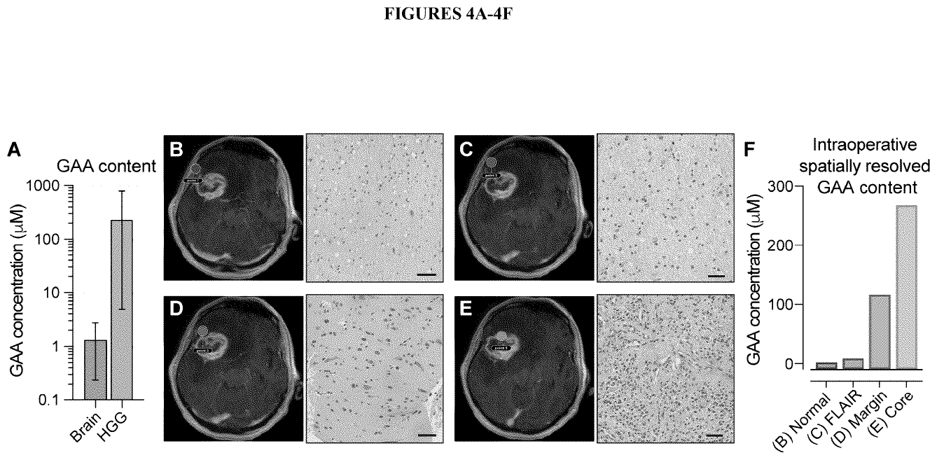

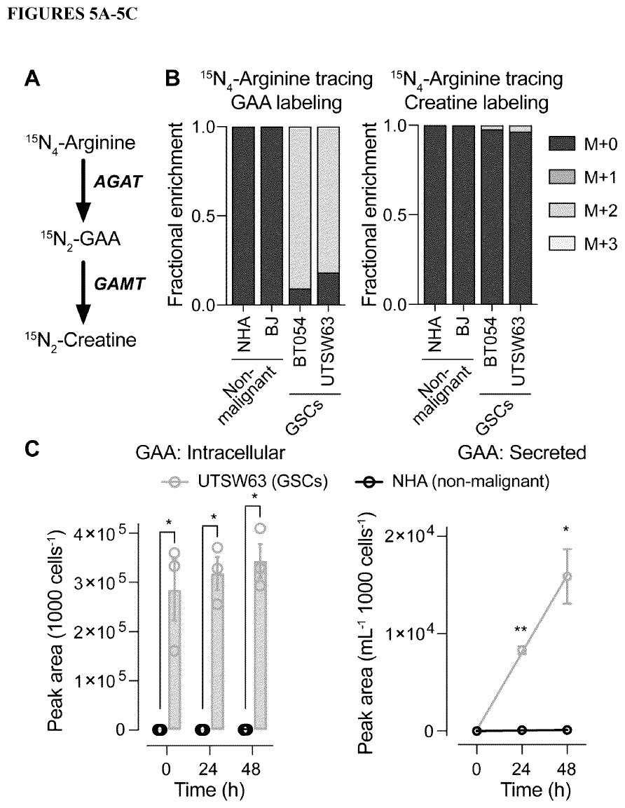

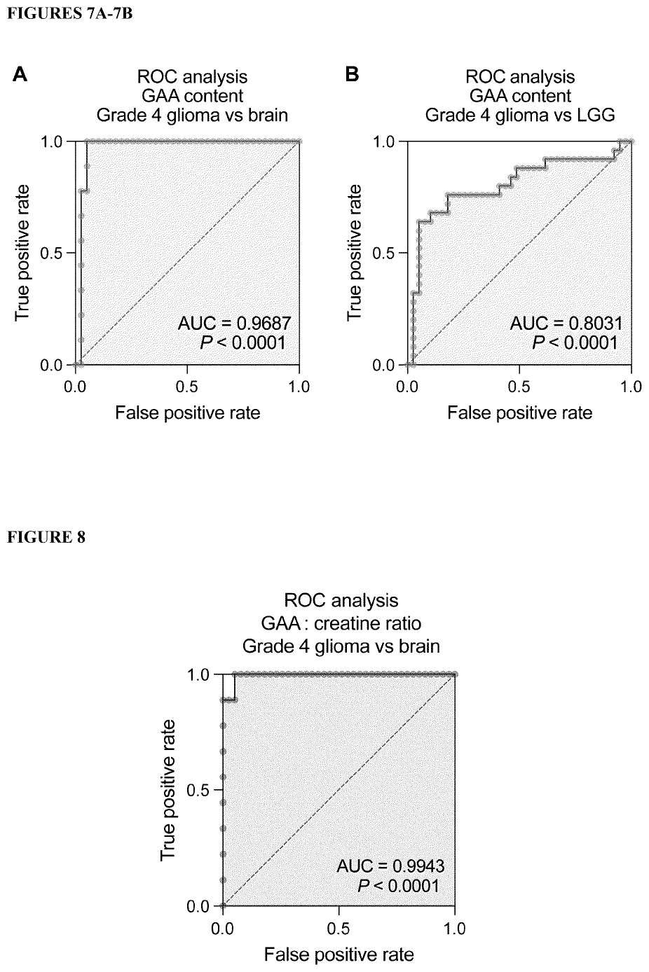

Next, an absolute quantification assay for GAA (FIG. 4A) was used to demonstrate that GAA specifically accumulates in the HGG microenvironment. Spatially defined tissue specimens collected intraoperatively during resection of a HGG (FIGS. 4B-4F) were evaluated. Results show that 1) HGGs display a bottleneck in the creatine synthesis pathway at the step of GAMT activity (FIG. 2B), and 2) HGGs engage in robust GAA synthesis that is uncoupled from its canonical role as a creatine precursor. ROC analyses using relative quantification data from the tissue cohort further showed that GAA is a robust biomarker of HGG. This is demonstrated using GAA content for HGGs versus non-malignant brain (FIG. 7A; p-value<0.0001) or HGGs versus LGGs (FIG. 7B; p-value<0.0001). This is additionally demonstrated using the GAA:creatine ratio for HGGs versus non-malignant brain (FIG. 8; p-value<0.0001).

GAA secretion is tumor-specific and associated with imbalanced AGAT and GAMT expression. GAA is upregulated ~100-fold versus relevant controls in human HGG brain tissues (FIGS. 3A and 4A). To determine whether this metabolic phenotype is preserved in glioma neurosphere models, ¹⁵N₄-arginine stable isotope tracing and metabolomics studies of glioma stem-like cell (GSC) neurosphere lines and non-malignant controls were performed. Arginine robustly labeled GAA but not creatine in GSCs at 18 hours, indicating that these cells display a bottleneck in the creatine synthesis pathway at the GAMT step (FIGS. 5A and 5B). The cells also exhibited high intracellular accumulation and secretion of GAA (FIG. 5C). In contrast, non-malignant cells, including NHA immortalized human astrocytes and BJ fibroblasts, showed no creatine synthesis pathway activity and did not accumulate or secrete GAA. Thus, cultured GSCs display a tumor-specific GAA overflow metabolism phenotype that aligns with findings from the metabolomics study of human brain tissues.

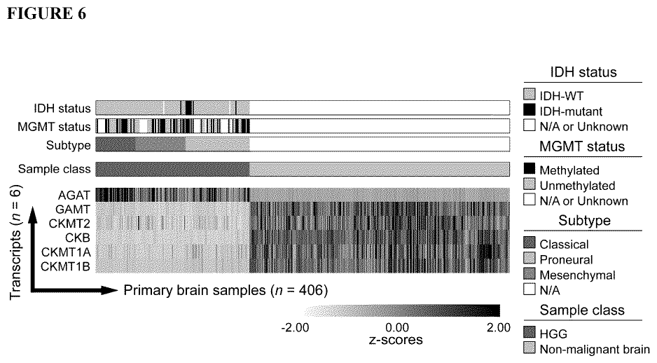

GAMT mutations are absent in HGGs. To address the mechanism underlying GAA overflow metabolism in HGGs, expression of creatine synthesis pathway genes in HGGs and non-malignant brain specimens were analyzed. AGAT expression was markedly upregulated in a subtype-independent manner in GBMs versus non-malignant controls, whereas the opposite pattern was observed for GAMT and creatine kinase transcripts (CKMT1A/B, CKMT2, and CKB) (FIG. 6). Therefore, GAA overflow metabolism in glioma is associated with imbalanced expression of AGAT and GAMT enzymes.

### Discussion

The role of peripheral biomarkers in HGG diagnosis and treatment has been an area of intense interest. HGGs, like other solid tumors, shed malignant cells and genomic DNA that can be detected in CSF or blood at low levels. However, biomarkers that can reliably differentiate inflammation, progression, or aggressiveness of HGG have not yet reached the threshold for widespread clinical use, partly due to the extremely high heterogeneity of HGGs which has hindered the development of universal disease biomarkers that can be applied across patients. These data exemplify GAA accumulation as a ubiquitous and tumor-specific feature of HGGs, including GBMs, which can be used to monitor HGG progression accurately and non-invasively.

Through metabolite profiling of human brain tissue specimens, GAA was found to be upregulated in a grade-dependent manner in glioma. These findings link GAA accumulation with brain tumor aggressiveness. GAA accumulation was determined using unbiased metabolomics analysis of 564 unique metabolites in 91 human tissues, comprising primary and metastatic brain tumors and non-malignant brain specimens. The inclusion of non-malignant brain specimens and metastases enhanced identification of novel glioma-specific metabolic phenotypes, including upregulation of GAA in HGGs.

The accumulation of GAA in HGG specimens relative to non-malignant brain tissue samples opens new avenues for enhancing surgical treatment of HGG. Specifically, defining tumor-brain margins intraoperatively is a challenge for HGG resection. On one hand, extent of resection is a major determinant of overall survival of patients with HGG. On the other hand, aggressive HGG resections that extend beyond the tumor-brain margin can damage critical brain structures, leading to permanent neurological impairment. Intraoperative mass spectrometry methods have been developed to survey tissue metabolite content in real time to assist neurosurgical oncologists in locating tumor-brain margins and achieving maximal safe glioma resection. However, these methods largely focus on detecting the metabolite 2-HG. These tumors, by definition, harbor IDH mutations that cause tumor-specific 2-HG accumulation. Although this is a compelling approach to improve resection of gliomas with IDH mutations, this approach is not useful for GBM resection surgeries because these tumors lack IDH mutations and harbor low levels of 2-HG. These results fill a critical gap in neurosurgical glioma care by nominating GAA as a tissue biomarker of GBM that can be exploited by intraoperative mass spectrometry to identify and navigate tumor margins during GBM resection surgeries.

### Example 2: GAA Quantification Assays for Biological Fluids

After tumor resection and chemoradiotherapy, patients undergo routine radiographic monitoring for relapse. Although radiographic changes can indicate tumor progression, radiographic changes can also be caused by pseudoprogression driven by treatment effects. As many as 50% of patients experience pseudoprogression resulting in radiographic changes. To make an accurate diagnosis, many patients undergo surgery to obtain a tissue diagnosis. There is need for methods of GAA quantification in CSF or other biological fluids, e.g., plasma or urine, which can be integrated into this workflow after radiographic changes are observed and before the decision to perform surgery. Craniotomies are highly invasive procedures and sparing patients from an additional surgery would be a major benefit. This is particularly advantageous for patients with recurrent glioma that are either too frail to tolerate an additional resection or whose tumor is inoperable; a definitive diagnosis of recurrent glioma by GAA quantification in a liquid biopsy sample can allow them to avoid a second surgery and provide rationale to initiate a new round of radiation therapy. Additionally, a definitive diagnosis of pseudoprogression by GAA quantification in a liquid biopsy sample can spare patients from undergoing a craniotomy to obtain a tissue specimen for assessment of disease recurrence.

### Results

A liquid chromatography-tandem mass spectrometry (LC-MS/MS) method was developed for absolute quantification of GAA in brain tissue, CSF, and plasma. To show feasibility, the method for measuring GAA was demonstrated in brain tissues (FIG. 4A). This assay was used to measure the GAA content of HGG and non-malignant human brain specimens, and recapitulated the ~100-fold increase in this metabolite that was observed in relative quantification liquid chromatography-mass spectrometry (LC-MS) metabolite profiling analyses (FIG. 3A). The absolute quantification assay for GAA is based on modification of a dried blood spot assay used to screen for GAMT deficiency in newborns in the states of Utah and New York. This method involves metabolite extraction from dried blood spots in methanol. GAA concentrations of extracts are measured via flow injection analysis electrospray ionization tandem mass spectrometry (FIA-ESI-MS/MS) by monitoring GAA precursor and product ions of 118.1 m/z and 76 m/z, respectively. The present method involves extracting tissue metabolites in 80% methanol with physical homogenization. Metabolite extracts are dried, reconstituted in 80% acetonitrile, and analyzed via LC-MS/MS using the same ion transition as the clinical assay: 118.1 m/z and 76 m/z. For both assays, isotopically labeled internal standards are included in solvents to enable assessments of extraction efficiency and instrument performance. Absolute GAA quantities are determined by comparing with a range of isotopically labeled GAA standards.

Key parameters related to assay performance, including the lower limit of quantitation and the linear range (calibration curve), intra-and-inter day accuracy and precision, recovery, matrix effects, and sample stability, have been determined. Precision and accuracy values were determined at varying concentrations. The calculated mean concentrations relative to spiked concentrations were used to express accuracy in % deviation. Means, standard deviations and coefficients of variation were calculated from the QC values and used to estimate the intra- and inter-day precision. Carry-over was evaluated using vials of blank mobile phase at several locations in the analysis set. Dilution analyses were performed using two-fold greater than the highest calibration standard. Aliquots of samples were diluted, e.g., 2, 3, and 5-fold, prior to analysis. Each dilution curve was processed multiple times each and back calculated against standard curves. Bench top, triplicate freeze/thaw, post-processing, and autosampler stability were tested using standard protocols for each sample type. Recovery from each tissue was assessed by analysis of extracted samples spiked at three QC concentration levels compared to samples spiked at the same concentrations that were not extracted. Replicate controls and spiked samples were used for each QC level to generate standard curves. Recovery was determined by comparing non-extracted QC samples with extracted QC samples. Measured concentrations of the non-extracted QC samples were defined as 100%. Quantitative matrix effect was determined by comparing QC samples to that of blanks containing the reconstitution solvent.

GAA quantification assays for brain tissue, CSF, and plasma were considered to be sensitive and specific at rates of 70% or above. This threshold reflects the sensitivity and specificity rates for the MGMT promotor methylation assay that is used to predict chemotherapy sensitivity in HGG, which is the most commonly used and widely accepted predictive assay in clinical use for glioma.

### REFERENCES

1. Marques E P, Wyse A T S. Guanidinoacetate Methyltransferase Deficiency: A Review of Guanidinoacetate Neurotoxicity. Journal of Inborn Errors of Metabolism and Screening. 2016 Jul. 29; 4:232640981666937.
2. Neu A, Neuhoff H, Trube G, Fehr S, Ullrich K, Roeper J, Isbrandt D. Activation of GABA(A) receptors by guanidinoacetate: a novel pathophysiological mechanism. Neurobiol Dis. 2002 November; 11(2):298-307. PMID: 12505422
3. Zugno A I, Scherer E B S, Schuck P F, Oliveira D L, Wofchuk S, Wannmacher C M D, Wajner M, Wyse A T S. Intrastriatal administration of guanidinoacetate inhibits Na+, K+-ATPase and creatine kinase activities in rat striatum. Metab Brain Dis. 2006 March; 21(1):41-50. PMID: 16773469
4. Zugno A I, Oliveira D L, Scherer E B S, Wajner M, Wofchuk S, Wyse A T S. Guanidinoacetate inhibits glutamate uptake in rat striatum of rats at different ages. Neurochem Res. 2007 June; 32(6):959-964. PMID: 17273928
5. Venkatesh H S, Morishita W, Geraghty A C, Silverbush D, Gillespie S M, Arzt M, Tam L T, Espenel C, Ponnuswami A, Ni L, Woo P J, Taylor K R, Agarwal A, Regev A, Brang D, Vogel H, Hervey-Jumper S, Bergles D E, Suvi M L, Malenka R C, Monje M. Electrical and synaptic integration of glioma into neural circuits. Nature. 2019 September; 573(7775):539-545. PMCID: PMC7038898
6. Venkatesh H S, Johung T B, Caretti V, Noll A, Tang Y, Nagaraja S, Gibson E M, Mount C W, Polepalli J, Mitra S S, Woo P J, Malenka R C, Vogel H, Bredel M, Mallick P, Monje M. Neuronal Activity Promotes Glioma Growth through Neuroligin-3 Secretion. Cell. 2015 May 7; 161(4):803-816. PMCID: PMC4447122
7. Venkatesh H S, Tam L T, Woo P J, Lennon J, Nagaraja S, Gillespie S M, Ni J, Duveau D Y, Morris P J, Zhao J J, Thomas C J, Monje M. Targeting neuronal activity-regulated neuroligin-3 dependency in high-grade glioma. Nature. 2017 Sep. 28; 549(7673):533-537. PMCID: PMC5891832
8. Venkataramani V, Tanev D I, Strahle C, Studier-Fischer A, Fankhauser L, Kessler T, Korber C, Kardorff M, Ratliff M, Xie R, Horstmann H, Messer M, Paik S P, Knabbe J, Sahm F, Kurz F T, Acikgoz A A, Herrmannsdorfer F, Agarwal A, Bergles D E, Chalmers A, Miletic H, Turcan S, Mawrin C, Hanggi D, Liu H K, Wick W, Winkler F, Kuner T. Glutamatergic synaptic input to glioma cells drives brain tumour progression. Nature. 2019 September; 573(7775):532-538. PMID: 31534219
9. Ostrom Q T, Price M, Neff C, Cioffi G, Waite K A, Kruchko C, Barnholtz-Sloan J S. CBTRUS Statistical Report: Primary Brain and Other Central Nervous System Tumors Diagnosed in the United States in 2015-2019. Neuro Oncol. 2022 Oct. 5; 24(Suppl 5):v1-v95. PMCID: PMC9533228
10. Louis D N, Perry A, Wesseling P, Brat D J, Cree I A, Figarella-Branger D, Hawkins C, Ng H K, Pfister S M, Reifenberger G, Soffietti R, von Deimling A, Ellison D W. The 2021 WHO Classification of Tumors of the Central Nervous System: a summary. Neuro Oncol. 2021 Aug. 2; 23(8):1231-1251. PMCID: PMC8328013
11. Liu R, Page M, Solheim K, Fox S, Chang S M. Quality of life in adults with brain tumors: current knowledge and future directions. Neuro Oncol. 2009 June; 11(3):330-339. PMCID: PMC2718978
12. Tan A C, Ashley D M, López GY, Malinzak M, Friedman HS, Khasraw M. Management of glioblastoma: State of the art and future directions. CA Cancer J Clin. 2020 July; 70(4):299-312. PMID: 32478924
13. DeBerardinis R J, Lum J J, Hatzivassiliou G, Thompson C B. The biology of cancer: metabolic reprogramming fuels cell growth and proliferation. Cell Metab. 2008 January; 7(1):11-20. PMID: 18177721
14. Hanahan D, Weinberg R A. Hallmarks of cancer: the next generation. Cell. 2011 Mar. 4; 144(5):646-674. PMID: 21376230
15. Vander Heiden M G, Cantley L C, Thompson C B. Understanding the Warburg effect: the metabolic requirements of cell proliferation. Science. 2009 May 22; 324(5930):1029-1033. PMCID: PMC2849637
16. Parsons D W, Jones S, Zhang X, Lin J C, Leary R J, Angenendt P, Mankoo P, Carter H, Siu I M, Gallia G L, Olivi A, McLendon R, Rasheed B A, Keir S, Nikolskaya T, Nikolsky Y, Busam D A, Tekleab H, Diaz L A, Hartigan J, Smith D R, Strausberg R L, Marie S K, Shinjo S M, Yan H, Riggins G J, Bigner D D, Karchin R, Papadopoulos N, Parmigiani G, Vogelstein B, Velculescu V E, Kinzler K W. An integrated genomic analysis of human glioblastoma multiforme. Science. 2008 Sep. 26; 321(5897):1807-12.
17. Yan H, Parsons D W, Jin G, McLendon R, Rasheed B A, Yuan W, Kos I, Batinic-Haberle I, Jones S, Riggins G J, Friedman H, Friedman A, Reardon D, Herndon J, Kinzler K W, Velculescu V E, Vogelstein B, Bigner D D. IDH1 and IDH2 mutations in gliomas. N Engl J Med. 2009 Feb. 19; 360(8):765-73.
18. Dang L, White D W, Gross S, Bennett B D, Bittinger M A, Driggers E M, Fantin V R, Jang H G, Jin S, Keenan M C, Marks K M, Prins R M, Ward P S, Yen K E, Liau L M, Rabinowitz J D, Cantley L C, Thompson C B, Vander Heiden M G, Su S M. Cancer-associated IDH1 mutations produce 2-hydroxyglutarate. Nature. 2009 Dec. 10; 462(7274):739-44.
19. Lu C, Ward P S, Kapoor G S, Rohle D, Turcan S, Abdel-Wahab O, Edwards C R, Khanin R, Figueroa M E, Melnick A, Wellen K E, O'Rourke D M, Berger S L, Chan T A, Levine R L, Mellinghoff I K, Thompson C B. IDH mutation impairs histone demethylation and results in a block to cell differentiation. Nature. 2012 Mar. 22; 483(7390):474-8.
20. Mellinghoff I K, Penas-Prado M, Peters K B, Burris H A, Maher E A, Janku F, Cote G M, de la Fuente M I, Clarke J L, Ellingson B M, Chun S, Young R J, Liu H, Choe S, Lu M, Le K, Hassan I, Steelman L, Pandya S S, Cloughesy T F, Wen P Y. Vorasidenib, a Dual Inhibitor of Mutant IDH1/2, in Recurrent or Progressive Glioma; Results of a First-in-Human Phase I Trial. Clin Cancer Res. 2021 Aug. 15; 27(16):4491-4499. PMCID: PMC8364866
21. Tateishi K, Wakimoto H, Iafrate A J, Tanaka S, Loebel F, Lelic N, Wiederschain D, Bedel O, Deng G, Zhang B, He T, Shi X, Gerszten R E, Zhang Y, Yeh J J, Curry W T, Zhao D, Sundaram S, Nigim F, Koerner M V A, Ho Q, Fisher D E, Roider E M, Kemeny L V, Samuels Y, Flaherty K T, Batchelor T T, Chi A S, Cahill D P. Extreme Vulnerability of IDH1 Mutant Cancers to NAD+ Depletion. Cancer Cell. 2015 Dec. 14; 28(6):773-784. PMCID: PMC4684594
22. Sulkowski P L, Corso C D, Robinson N D, Scanlon S E, Purshouse K R, Bai H, Liu Y, Sundaram R K, Hegan D C, Fons N R, Breuer G A, Song Y, Mishra-Gorur K, De Feyter H M, de Graaf R A, Surovtseva Y V, Kachman M, Halene S, Gunel M, Glazer P M, Bindra R S. 2-Hydroxyglutarate produced by neomorphic IDH mutations suppresses homologous recombination and induces PARP inhibitor sensitivity. Sci Transl Med. 2017 Feb. 1; 9(375). PMCID: PMC5435119
23. Sulkowski P L, Oeck S, Dow J, Economos N G, Mirfakhraie L, Liu Y, Noronha K, Bao X, Li J, Shuch B M, King M C, Bindra R S, Glazer P M. Oncometabolites suppress DNA repair by disrupting local chromatin signalling. Nature. 2020 June; 582(7813):586-591. PMCID: PMC7319896
24. McBrayer S K, Mayers J R, DiNatale G J, Shi D D, Khanal J, Chakraborty A A, Sarosiek K A, Briggs K J, Robbins A K, Sewastianik T, Shareef S J, Olenchock B A, Parker S J, Tateishi K, Spinelli J B, Islam M, Haigis M C, Looper R E, Ligon K L, Bernstein B E, Carrasco R D, Cahill D P, Asara J M, Metallo C M, Yennawar N H, Vander Heiden M G, Kaelin W G. Transaminase Inhibition by 2-Hydroxyglutarate Impairs Glutamate Biosynthesis and Redox Homeostasis in Glioma. Cell. 2018 Sep. 20; 175(1):101-116 e25.
25. Kizilbash S, Piccioni D, Sahebjam S, Villano J, Jaeckle K, Welch M, Kong X T, Holdhoff M, Lammers A, Remick A, Reid J, Allred J, Port J, Lanza I, McBrayer S, Kunos C, Shapiro G, Adjei A. CTNI-23. Preliminary Safety And Pharmacokinetics Data For A Phase 1b Trial Of Telaglenastat In Combination With Radiation Therapy And Temozolomide In Patients With Idh-Mutant Grade 2/3 Astrocytoma (NCI-10218). Neuro-Oncology. 2022 Nov. 14; 24(Supplement_7):vii75-vii75.
26. Shi D D, Savani M R, Levitt M M, Wang A C, Endress J E, Bird C E, Buehler J, Stopka S A, Regan M S, Lin Y F, Puliyappadamba V T, Gao W, Khanal J, Evans L, Lee J H, Guo L, Xiao Y, Xu M, Huang B, Jennings R B, Bonal D M, Martin-Sandoval M S, Dang T, Gattie L C, Cameron A B, Lee S, Asara J M, Kornblum H I, Mak T W, Looper R E, Nguyen Q D, Signoretti S, Gradl S, Sutter A, Jeffers M, Janzer A, Lehrman M A, Zacharias L G, Mathews T P, Losman J A, Richardson T E, Cahill D P, DeBerardinis R J, Ligon K L, Xu L, Ly P, Agar NYR, Abdullah K G, Harris I S, Kaelin W G, McBrayer S K. De novo pyrimidine synthesis is a targetable vulnerability in IDH mutant glioma. Cancer Cell. 2022 Sep. 12; 40(9):939-956.e16. PMCID: PMC9515386
27. Chinnaiyan P, Kensicki E, Bloom G, Prabhu A, Sarcar B, Kahali S, Eschrich S, Qu X, Forsyth P, Gillies R. The metabolomic signature of malignant glioma reflects accelerated anabolic metabolism. Cancer Res. 2012 Nov. 15; 72(22):5878-5888. PMCID: PMC4638418
28. Prabhu A H, Kant S, Kesarwani P, Ahmed K, Forsyth P, Nakano I, Chinnaiyan P. Integrative cross-platform analyses identify enhanced heterotrophy as a metabolic hallmark in glioblastoma. Neuro Oncol. 2019 Feb. 19; 21(3):337-347. PMCID: PMC6380409
29. Wang L B, Karpova A, Gritsenko M A, Kyle J E, Cao S, Li Y, Rykunov D, Colaprico A, Rothstein J H, Hong R, Stathias V, Cornwell M, Petralia F, Wu Y, Reva B, Krug K, Pugliese P, Kawaler E, Olsen L K, Liang W W, Song X, Dou Y, Wendl M C, Caravan W, Liu W, Cui Zhou D, Ji J, Tsai C F, Petyuk V A, Moon J, Ma W, Chu R K, Weitz K K, Moore R J, Monroe M E, Zhao R, Yang X, Yoo S, Krek A, Demopoulos A, Zhu H, Wyczalkowski M A, McMichael J F, Henderson B L, Lindgren C M, Boekweg H, Lu S, Baral J, Yao L, Stratton K G, Bramer L M, Zink E, Couvillion S P, Bloodsworth K J, Satpathy S, Sieh W, Boca S M, Schürer S, Chen F, Wiznerowicz M, Ketchum K A, Boja E S, Kinsinger C R, Robles A I, Hiltke T, Thiagarajan M, Nesvizhskii A I, Zhang B, Mani D R, Ceccarelli M, Chen X S, Cottingham S L, Li Q K, Kim A H, Fenyo D, Ruggles K V, Rodriguez H, Mesri M, Payne S H, Resnick A C, Wang P, Smith R D, Iavarone A, Chheda M G, Barnholtz-Sloan J S, Rodland K D, Liu T, Ding L, Clinical Proteomic Tumor Analysis Consortium. Proteogenomic and metabolomic characterization of human glioblastoma. Cancer Cell. 2021 Apr. 12; 39(4):509-528.e20. PMCID: PMC8044053
30. Scott A J, Correa L O, Edwards D, Sun Y, Ravikumar V, Andren A C, Zhang L, Srinivasan S, Jairath N, Verbal K, Muraszko K, Sagher O, Carty S A, Hervey-Jumper S, Orringer D, Kim M M, Junck L, Umemura Y, Leung D, Venneti S, Camelo-Piragua S, Lawrence T S, Ippolito J E, Al-Holou W N, Chinnaiyan P, Heth J, Rao A, Lyssiotis C A, Wahl D R. Metabolomic profiles of human glioma inform patient survival. Antioxid Redox Signal. 2023 Feb. 27; PMID: 36852494
31. Verhaak R G, Hoadley K A, Purdom E, Wang V, Qi Y, Wilkerson M D, Miller C R, Ding L, Golub T, Mesirov J P, Alexe G, Lawrence M, O'Kelly M, Tamayo P, Weir B A, Gabriel S, Winckler W, Gupta S, Jakkula L, Feiler H S, Hodgson J G, James C D, Sarkaria I N, Brennan C, Kahn A, Spellman P T, Wilson R K, Speed T P, Gray J W, Meyerson M, Getz G, Perou C M, Hayes D N. Integrated genomic analysis identifies clinically relevant subtypes of glioblastoma characterized by abnormalities in PDGFRA, IDH1, EGFR, and NFl. Cancer Cell. 2010 Jan. 19; 17(1):98-110.
32. Humm A, Fritsche E, Steinbacher S. Structure and reaction mechanism of L-arginine:glycine amidinotransferase. Biol Chem. 1997; 378(3-4):193-197. PMID: 9165070
33. Florian C L, Preece N E, Bhakoo K K, Williams S R, Noble M. Characteristic metabolic profiles revealed by 1H NMR spectroscopy for three types of human brain and nervous system tumours. NMR Biomed. 1995 September; 8(6):253-264. PMID: 8732181
34. Daly M M. Guanidinoacetate methyltransferase activity in tissues and cultured cells. Arch Biochem Biophys. 1985 Feb. 1; 236(2):576-584. PMID: 3970526
35. Wyss M, Kaddurah-Daouk R. Creatine and creatinine metabolism. Physiol Rev. 2000 July; 80(3):1107-1213. PMID: 10893433
36. Schulze A. Creatine deficiency syndromes. Mol Cell Biochem. 2003 February; 244(1-2):143-150. PMID: 12701824
37. D′Hooge R, Pei Y Q, Marescau B, De Deyn P P. Convulsive action and toxicity of uremic guanidino compounds: behavioral assessment and relation to brain concentration in adult mice. J Neurol Sci. 1992 October; 112(1-2):96-105. PMID: 1469446
38. Stern W M, Winston J S, Murphy E, Cross J H, Sander J W. Guanidinoacetate methyltransferase (GAMT) deficiency: a rare but treatable epilepsy. Pract Neurol. 2017 June; 17(3):207-211. PMID: 28119378
39. Schulze A, Ebinger F, Rating D, Mayatepek E. Improving treatment of guanidinoacetate methyltransferase deficiency: reduction of guanidinoacetic acid in body fluids by arginine restriction and ornithine supplementation. Mol Genet Metab. 2001 December; 74(4):413-419. PMID: 11749046
40. Krishna S, Choudhury A, Keough M B, Seo K, Ni L, Kakaizada S, Lee A, Aabedi A, Popova G, Lipkin B, Cao C, Nava Gonzales C, Sudharshan R, Egladyous A, Almeida N, Zhang Y, Molinaro A M, Venkatesh H S, Daniel A G S, Shamardani K, Hyer J, Chang E F, Findlay A, Phillips J J, Nagarajan S, Raleigh D R, Brang D, Monje M, Hervey-Jumper S L. Glioblastoma remodelling of human neural circuits decreases survival. Nature. 2023 May 3; PMID: 37138086
41. Zhao H, Heimberger A B, Lu Z, Wu X, Hodges T R, Song R, Shen J. Metabolomics profiling in plasma samples from glioma patients correlates with tumor phenotypes. Oncotarget. 2016 Apr. 12; 7(15):20486-20495. PMCID: PMC4991469
42. Riviere-cazaux C, Carlstrom L P, Rajani K, Munoz-casabella A, Rahman M, Gharibi-Loron A, Brown D A, Miller K J, White J J, Himes B T, Jusue-Torres I, Ikram S, Ransom S, Hirte R, Oh J H, Elmquist W F, Sarkaria J N, Vaubel R A, Rodriguez M, Warrington A, Kizilbash S H, Burns T C. Does Blood-Brain Barrier Disruption Define the Glioma Extracellular Metabolome?[Internet]. Oncology; 2021 August. Available from: http://medrxiv.org/lookup/doi/10.1101/2021.08.24.21262320
43. Stockler-Ipsiroglu S, van Karnebeek C, Longo N, Korenke G C, Mercimek-Mahmutoglu S, Marquart I, Barshop B, Grolik C, Schlune A, Angle B, Aranjo H C, Coskun T, Diogo L, Geraghty M, Haliloglu G, Konstantopoulou V, Leuzzi V, Levtova A, Mackenzie J, Maranda B, Mhanni A A, Mitchell G, Morris A, Newlove T, Renaud D, Scaglia F, Valayannopoulos V, van Spronsen F J, Verbruggen K T, Yuskiv N, Nyhan W, Schulze A. Guanidinoacetate methyltransferase (GAMT) deficiency: outcomes in 48 individuals and recommendations for diagnosis, treatment and monitoring. Mol Genet Metab. 2014 January; 111(1):16-25. PMID: 24268530
44. Schmidt A, Marescau B, Boehm E A, Renema W K J, Peco R, Das A, Steinfeld R, Chan S, Wallis J, Davidoff M, Ullrich K, Waldschütz R, Heerschap A, De Deyn P P, Neubauer S, Isbrandt D. Severely altered guanidino compound levels, disturbed body weight homeostasis and impaired fertility in a mouse model of guanidinoacetate N-methyltransferase (GAMT) deficiency. Hum Mol Genet. 2004 May 1; 13(9):905-921. PMID: 15028668
45. Caldeira Aranjo H, Smit W, Verhoeven N M, Salomons G S, Silva S, Vasconcelos R, Tomis H, Tavares de Almeida I, Jakobs C, Duran M. Guanidinoacetate methyltransferase deficiency identified in adults and a child with mental retardation. Am J Med Genet A. 2005 Mar. 1; 133A(2):122-127. PMID: 15651030
46. Mortazavi A, Fayed I, Bachani M, Dowdy T, Jahanipour J, Khan A, Owotade J, Walbridge S, Inati S K, Steiner J, Wu J, Gilbert M, Yang C Z, Larion M, Maric D, Ksendzovsky A, Zaghloul K A. IDH-mutated gliomas promote epileptogenesis through d-2-hydroxyglutarate-dependent mTOR hyperactivation. Neuro Oncol. 2022 Sep. 1; 24(9):1423-1435. PMCID: PMC9435503
47. Gross M D, Eggen M A, Simon A M, Van Pilsum J F. The purification and characterization of human kidney L-arginine:glycine amidinotransferase. Arch Biochem Biophys. 1986 December; 251(2):747-755. PMID: 3800397
48. Bianchi M, Rossi L, Pierige F, De Angeli P, Aliano M P, Carducci C, Di Carlo E, Pascucci T, Nardecchia F, Leuzzi V, Magnani M. Engineering new metabolic pathways in isolated cells for the degradation of guanidinoacetic acid and simultaneous production of creatine. Mol Ther Methods Clin Dev. 2022 Jun. 9; 25:26-40. PMCID: PMC8917272
49. Abdullah K G, Bird C E, Buehler J D, Gattie L C, Savani M R, Sternisha A C, Xiao Y, Levitt M M, Hicks W H, Li W, Ramirez D M O, Patel T, Garzon-Muvdi T, Barnett S, Zhang G, Ashley D M, Hatanpaa K J, Richardson T E, McBrayer S K. Establishment of patient-derived organoid models of lower-grade glioma. Neuro Oncol. 2022 Apr. 1; 24(4):612-623. PMCID: PMC8972292
50. Buehler J D, Bird C E, Savani M R, Gattie L C, Hicks W H, Levitt M M, El Shami M, Hatanpaa K J, Richardson T E, McBrayer S K, Abdullah K G. Semi-Automated Computational Assessment of Cancer Organoid Viability Using Rapid Live-Cell Microscopy. Cancer Inform. 2022; 21:11769351221100754. PMCID: PMC9150230
51. Yu S, Wei S, Savani M, Lin X, Du K, Mender I, Siteni S, Vasilopoulos T, Reitman Z J, Ku Y, Wu D, Liu H, Tian M, Chen Y, Labrie M, Charbonneau C M, Sugarman E, Bowie M, Hariharan S, Waitkus M, Jiang W, McLendon R E, Pan E, Khasraw M, Walsh K M, Lu Y, Herlyn M, Mills G, Herbig U, Wei Z, Keir S T, Flaherty K, Liu L, Wu K, Shay J W, Abdullah K, Zhang G, Ashley D M. A Modified Nucleoside 6-Thio-2′-Deoxyguanosine Exhibits Antitumor Activity in Gliomas. Clin Cancer Res. 2021 Dec. 15; 27(24):6800-6814. PMCID: PMC8678347
52. Cantor J R, Abu-Remaileh M, Kanarek N, Freinkman E, Gao X, Louissaint A, Lewis C A, Sabatini D M. Physiologic Medium Rewires Cellular Metabolism and Reveals Uric Acid as an Endogenous Inhibitor of UMP Synthase. Cell. 2017 Apr. 6; 169(2):258-272.e17. PMCID: PMC5421364
53. El Shami M, Savani M R, Gattie L C, Smith B, Hicks W H, Rich I N, Richardson T E, McBrayer S K, Abdullah K G. Human plasma-like medium facilitates metabolic tracing and enables upregulation of immune signaling pathways in glioblastoma explants. bioRxiv [Internet]. 2023; Available from: https://www.biorxiv.org/content/10.1101/2023.05.29.542774v1
54. Stockler-Ipsiroglu S, Apatean D, Battini R, DeBrosse S, Dessoffy K, Edvardson S, Eichler F, Johnston K, Koeller D M, Nouioua S, Tazir M, Verma A, Dowling M D, Wierenga K J, Wierenga A M, Zhang V, Wong L J C. Arginine:glycine amidinotransferase (AGAT) deficiency: Clinical features and long term outcomes in 16 patients diagnosed worldwide. Mol Genet Metab. 2015 December; 116(4):252-259. PMID: 26490222
55. Muenchhoff J, Siddiqui K S, Poljak A, Raftery M J, Barrow K D, Neilan B A. A novel prokaryotic L-arginine:glycine amidinotransferase is involved in cylindrospermopsin biosynthesis. FEBS J. 2010 September; 277(18):3844-3860. PMID: 20718864
56. Yasuhara M, Fujita S, Arisue K, Kohda K, Hayashi C. A new enzymatic method to determine creatine. Clin Chim Acta. 1982 Jul. 1; 122(2):181-188. PMID: 7105406
57. Nguyen T P, Wang W, Sternisha A C, Corley C D, Wang H Y L, Wang X, Ortiz F, Lim S K, Abdullah K G, Parada L F, Williams N S, McBrayer S K, McDonald J G, De Brabander J K, Nijhawan D. Selective and brain-penetrant lanosterol synthase inhibitors target glioma stem-like cells by inducing 24(S),25-epoxycholesterol production. Cell Chem Biol. 2023 Feb. 16; 30(2):214-229.e18. PMCID: PMC10008516
58. Rohle D, Popovici-Muller J, Palaskas N, Turcan S, Grommes C, Campos C, Tsoi J, Clark O, Oldrini B, Komisopoulou E, Kunii K, Pedraza A, Schalm S, Silverman L, Miller A, Wang F, Yang H, Chen Y, Kernytsky A, Rosenblum M K, Liu W, Biller S A, Su S M, Brennan C W, Chan T A, Graeber T G, Yen K E, Mellinghoff I K. An Inhibitor of Mutant IDH1 Delays Growth and Promotes Differentiation of Glioma Cells. Science. 2013; 340(6132):626-30. PMCID: PMC3985613
59. Chen T W, Wardill T J, Sun Y, Pulver S R, Renninger S L, Baohan A, Schreiter E R, Kerr R A, Orger M B, Jayaraman V, Looger L L, Svoboda K, Kim D S. Ultrasensitive fluorescent proteins for imaging neuronal activity. Nature. 2013 Jul. 18; 499(7458):295-300. PMCID: PMC3777791
60. Grzywa T_{M}, Sosnowska A, Matryba P, Rydzynska Z, Jasinski M, Nowis D, Golab J. Myeloid Cell-Derived Arginase in Cancer Immune Response. Front Immunol. 2020; 11:938. PMCID: PMC7242730
61. Venkataramani V, Yang Y, Schubert M C, Reyhan E, Tetzlaff S K, Wilβmann N, Botz M, Soyka S J, Beretta C A, Pramatarov R L, Fankhauser L, Garofano L, Freudenberg A, Wagner J, Tanev D I, Ratliff M, Xie R, Kessler T, Hoffmann D C, Hai L, Dorflinger Y, Hoppe S, Yabo Y A, Golebiewska A, Niclou S P, Sahm F, Lasorella A, Slowik M, Doring L, Iavarone A, Wick W, Kuner T, Winkler F. Glioblastoma hijacks neuronal mechanisms for brain invasion. Cell. 2022 Aug. 4; 185(16):2899-2917.e31. PMID: 35914528
62. Kan H E, Meeuwissen E, van Asten J J, Veltien A, Isbrandt D, Heerschap A. Creatine uptake in brain and skeletal muscle of mice lacking guanidinoacetate methyltransferase assessed by magnetic resonance spectroscopy. J Appl Physiol (1985). 2007 June; 102(6):2121-2127. PMID: 17347380
63. Kurata K, Nagasawa M, Tomonaga S, Aoki M, Morishita K, Denbow D M, Furuse M. Orally administered L-ornithine elevates brain L-ornithine levels and has an anxiolytic-like effect in mice. Nutr Neurosci. 2011 November; 14(6):243-248. PMID: 22053755
64. Jomura R, Akanuma S I, Tachikawa M, Hosoya K I. SLC6A and SLC16A family of transporters: Contribution to transport of creatine and creatine precursors in creatine biosynthesis and distribution. Biochim Biophys Acta Biomembr. 2022; 1864(3):183840. Epub 20211216. doi: 10.1016/j.bbamem.2021.183840. PubMed PMID: 34921896.
65. Leuzzi V, Bianchi M C, Tosetti M, Carducci C, Cerquiglini C A, Cioni G, et al. Brain creatine depletion: guanidinoacetate methyltransferase deficiency (improving with creatine supplementation). Neurology. 2000; 55(9):1407-9. doi: 10.1212/wnl.55.9.1407. PubMed PMID: 11087795.
66. Iyamu E W, Asakura T, Woods G M. A colorimetric microplate assay method for high-throughput analysis of arginase activity in vitro. Anal Biochem. 2008; 383(2):332-4. Epub 20080823. doi: 10.1016/j.ab.2008.08.016. PubMed PMID: 18789882; PubMed Central PMCID: PMC2605851.
67. Takata Y, Konishi K, Gomi T, Fujioka M. Rat guanidinoacetate methyltransferase. Effect of site-directed alteration of an aspartic acid residue that is conserved across most mammalian S-adenosylmethionine-dependent methyltransferases. J Biol Chem. 1994; 269(8):5537-42. PubMed PMID: 8119887.
68. Kan H E, Meeuwissen E, Veltien A, Isbrandt D, Heerschap A. Creatine uptake kinetics in brain and skeletal muscle of GAMT deficient knockout mice. Proc Intl Soc Mag Reson Med 14; Seattle, WA, USA2006. p. 1484.
69. Kerstetter-Fogle A E, Harris P L R, Brady-Kalnay S M, Sloan A E. Generation of Glioblastoma Patient-Derived Intracranial Xenografts for Preclinical Studies. Int J Mol Sci. 2020; 21(14). Epub 20200720. doi: 10.3390/ijms21145113. PubMed PMID: 32698368; PubMed Central PMCID: PMC7403971.
70. Fletcher M, Ramirez M E, Sierra R A, Raber P, Thevenot P, Al-Khami A A, et al. 1-Arginine depletion blunts antitumor T-cell responses by inducing myeloid-derived suppressor cells. Cancer Res. 2015; 75(2):275-83. Epub 20141118. doi: 10.1158/0008-5472.Can-14-1491. PubMed PMID: 25406192; PubMed Central PMCID: PMC4297565.
71. Riviere-Cazaux C, Carlstrom L P, Rajani K, Munoz-Casabella A, Sarkaria J N, Rodriguez M, et al. Individualized diversity in the extracellular metabolome of live human gliomas. medRxiv 2021.08.24.21262320; doi: https://doi.org/10.1101/2021.08.24.21262320.
72. Riviere-Cazaux C, Carlstrom L P, Rajani K, Munoz-Casabella A, Rahman M, Gharibi-Loron A, et al. Does Blood-Brain Barrier Disruption Define the Glioma Extracellular Metabolome?medRxiv 2021.08.24.21262320; doi: https://doi.org/10.1101/2021.08.24.21262320.
73. Zhao H, Heimberger A B, Lu Z, Wu X, Hodges T R, Song R, et al. Metabolomics profiling in plasma samples from glioma patients correlates with tumor phenotypes. Oncotarget 2015; 7(15):20486-20495. PubMed PMID: 26967252.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and compositions of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps. Various patents, patent applications, publications, product descriptions, protocols, and sequence accession numbers are cited throughout this application, this present disclosures of which are incorporated herein by reference in their entireties for all purposes.

What is claimed is:

1. A method for resecting tumor tissue in a subject in need thereof, comprising:
a) collecting one or more samples from a suspected tumor tissue;
b) determining the level of guanidinoacetic acid (GAA) in the one or more samples;
c) comparing the level of GAA in the one or more samples to a reference level;
d) defining the suspected tumor tissue as tumor based on the level of GAA in the one or more samples; and
e) resecting the suspected tumor tissue from the subject where the suspected tumor tissue is defined as tumor.

2. The method of claim 1, wherein the level of GAA in the one or more samples is determined using a mass spectrometry method.

3. The method of claim 2, wherein the mass spectrometry method comprises mass spectrometry (MS), liquid chromatography-mass spectrometry (LC-MS) or liquid chromatography with tandem mass spectrometry (LC-MS/MS).

4. The method of claim 1, wherein the level of GAA in the one or more samples is determined using a non-invasive method.

5. The method of claim 4, wherein the non-invasive method comprises magnetic resonance spectroscopy, with a singlet peak in the region of 3.78 ppm, or as a ratio between the GAA peak and the nearby Cr peak at 3.19 ppm.

6. The method of claim 1, wherein the reference level is the amount of GAA in a non-tumor tissue sample.

7. The method of claim 1, wherein the tumor is a glioma.

8. The method of claim 7, wherein the glioma is a high-grade glioma (HGG), a glioblastoma multiforme or an astrocytoma.

9. The method of claim 7, wherein the glioma is a Grade 3 glioma.

10. The method of claim 7, wherein the glioma is a Grade 4 glioma.

11. The method of claim 7, wherein the glioma is a recurrent glioma.

12. The method of claim 7, wherein the glioma is an adult glioma, a pediatric glioma, a pediatric astrocytoma, a pediatric oligodendroglioma, a pediatric diffuse midline glioma, or a pediatric diffuse hemispheric glioma.

13. The method of claim 1, wherein the one or more samples comprise tumor cells of the subject.

14. The method of claim 1, wherein the one or more samples comprise non-tumor cells of the subject.

15. The method of claim 1, further comprising determining the level of a second metabolite in the one or more samples selected from the group consisting of creatine, phosphocreatine, and creatinine, and comparing the level of the second metabolite in the one or more samples to a second reference level.

16. The method of claim 15, wherein the second reference level is the amount of the second metabolite in a non-tumor tissue sample.

17. The method of claim 15, further comprising comparing the ratio of GAA to the second metabolite in the one or more samples to a reference ratio.

18. The method of claim 17, wherein the reference ratio is the ratio of GAA to the second metabolite in a non-tumor tissue sample.

19. The method of claim 15, wherein the second metabolite is creatine.

20. The method of claim 1, wherein a tumor margin is defined based on the level of GAA in the one or more sample, and the tumor is resected based on the tumor margin.
